(19) United States
(12) Patent Application Publication
(10) Pub. No.: US 2023/0012231 A1
(43) Pub. Date: Jan. 12, 2023

(54) A METHOD AND APPARATUS FOR RESPIRATORY SECRETION COLLECTION AND ANALYSIS

(71) Applicant: Diag-Nose Medical Pty Ltd, Salter Point, Western Australia (AU)

(72) Inventors: Shaikh Salah El Din ROSTOM, Salter Point (AU); Josie XU, Toronto (CA); Brian Shih-ning WANG, Houston, TX (US); Jonathan LIMPAH, Salter Point (AU); David Melicio YEN, Bethlehem, PA (US); Aaron NOTT, Cremorne (AU); Daniel BOOKER, Cremorne (AU)

(73) Assignee: Diag-Nose Medical Pty Ltd, Salter Point, Western Australia (AU)

(21) Appl. No.: 17/782,581

(22) PCT Filed: Dec. 4, 2020
(86) PCT No.: PCT/IB2020/001000

§ 371 Date: Jun. 3, 2022

(30) Foreign Application Priority Data
Dec. 4, 2019 (AU) 2019904597
Jul. 2, 2020 (AU) 2020902259

(51) Int. Cl. A61B 10/00; G01N 33/543

(52) U.S. Cl. CPC A61B 10/0051; G01N 33/54366

(57) ABSTRACT

The invention relates to a respiratory secretion sample collection device that includes a collection reservoir for directly receiving a sample of a respiratory secretion, a displacement member for insertion into the collection reservoir and displacing the sample within the collection reservoir, a container of a diluent for fluid communication with the sample for mixing the diluent with the sample, and an outlet for discharging the mixture of the diluent and the sample to an assay device. In embodiments, the precise volumes of the sample and the diluent are effectively mixed to enable the conduct of assays for which the relative concentration of diluent and sample is critical.

### TECHNICAL FIELD

The present invention relates to the devices and methods for the collection of respiratory secretion samples for subsequent analysis, including nasal secretion samples. Aspects of the invention also relate to the assay kits detection of bacterial infections of the sinus cavity, or a viral respiratory infection or a fungal infection, or an allergic immune response or an inflammatory response. The samples include nasal secretions. However, it is to be appreciated that the invention may have broader application in relation to other samples such as sputum.

### BACKGROUND

Sinusitis is inflammation of the mucous membranes of the sinus cavities. Symptoms associated with sinusitis include inflammation, mucous discharge, blockage of the nasal cavity and facial pain. Other associated symptoms may include fever, headaches, loss of smell, sore throat, and a cough. The most common causes of sinusitis are viral infection, bacterial infection or and allergic immune response such as hay fever or a fungal infection. A symptom of sinusitis can include an inflammatory response indicative of chronic sinusitis, neurogenic rhinitis, non-infectious and non-allergic rhinitis.

Cold viruses are generally untreatable with pharmaceuticals. Nevertheless, antibiotics are often prescribed for the treatment of sinusitis and other symptoms associated with the common cold. However, such treatment is useless if there is no bacterial infection present. Accordingly, treatment of sinusitis is one of the major causes of oversubscription of antibiotics and is a leading contributor to the problem of antibiotic resistant bacteria.

Nevertheless, sometimes a secondary bacterial infection can occur that may require treatment with antibiotics. In some rare cases, particularly in people with weakened immune systems, a fungal infection can be the cause of sinusitis.

Sinusitis is a common pathology experienced by humans. Nevertheless, accurate diagnosis of the cause of the sinusitis remains a challenge. When a patient presents with sinusitis, the typical examination includes inspection of the face, ears, nose, throat and neck. The specialty nasal examination is performed with a headlight or mirror and a handheld speculum for examining the nasal septum and turbinates. A specialist may also examine the back of the nose using hand-held mirrors in the mouth.

There are a number of additional methods for further examination of sinusitis. One method includes nasal endoscopy which is where a thin, flexible tube with a fiber-optic light is inserted through a nostril and into the sinus cavity to visually inspect the sinuses. This technique can identify abnormal swelling, enlarged turbinates, nasal polyps, septal deformities, and sinus drainage.

Another method includes imaging studies through CT scans which may help find abnormalities or suspected complications. This approach is commonly used to document chronic sinusitis and to evaluate the potential benefit of surgical treatment of the chronic sinusitis.

If the cause of the sinusitis is suspected as being due to an allergy then an allergy skin test may be conducted to understand if an allergen is the cause.

If the cause of the sinusitis is suspected as being viral or bacterial, then laboratory tests may be employed. Laboratory testing is usually only employed if the sinusitis remains persistent or fails to respond to treatment with antibiotics or is worsening.

Tissue samples or cultures from the nose or sinuses can be employed to identify bacterial infection as the cause. The most common method for obtaining samples or cultures from the through, nose or sinuses includes an oropharyngeal and/or nasopharyngeal swab. The swab is inserted into the patient's nose and is aimed in a parallel direction to the nasal and septum floor. As long as there are no obstructions present within the nasal cavity, the swab will continue to move in this direction until it reaches the nasopharynx. Once the swab reaches the nasopharynx, it is rotated to allow the secretions from this area to be absorbed. Once the swab is removed from the patient's nose, it is immediately inserted into a vial that contains culture media. The swab handle that extends past the opening of the vial is snapped off to allow the tube to be closed.

Nasopharyngeal swabs are invasive and uncomfortable for the patient and trained medical personnel are required to carry out the swab.

Better cultures are taken directly from the sinus cavity during nasal endoscopy. The endoscopic observation of pus confirms the diagnosis of sinusitis and allows the specialist to place a small swab directly into the abnormal mucous. Culture specimens are sent to a laboratory and results may take up to a week. Endoscopic observation is also very invasive and uncomfortable and may require the patient to be sedated.

Another technique for obtaining a sinus culture is maxillary sinus puncture and aspiration. However, this technique is very invasive and requires puncturing the sinus membrane with a needle and taking an aspirate. This technique also requires administration of a local anaesthetic.

Testing of samples or cultures from the nose or sinuses is typically conducted in a laboratory and it can take some days for the physician and patient to receive the results.

Point of care blood-based tests have been developed to quickly indicate the presence or absence of viral infection. Such blood-based tests are relatively invasive and require a needle prick to extract a sample of the patient's blood. However, many patients are averse to needles including small children, pregnant women and haemophiliacs. Also, patients can be averse to conducting any diagnostic tests requiring the drawing of blood outside of a clinical environment, for example in the home or in a nursing home.

Home or point of care diagnostic kits have been developed for obtaining samples and conducting a diagnostic test to indicate a viral infection or a bacterial sinus infection. However, such diagnostic kits can be complex and difficult to use. For example, in the case of blood based kits, the user is presented with a collection of components, including the test device itself, a separate lancet and blood collection receptacle, a container of buffer or a test fluid and possibly more components. The user must follow a very specific set of instructions to carry out the test and to interpret the results. If the user does not frequently use such devices then the chance of misuse can be high.

More recently, there have been developments in blood-based kits that are self-contained and that afford less opportunity for misuse. For example, U.S. Pat. No. 10,525,463 discloses an integrated testing device and method including a body including an integrated lancet for obtaining a blood sample, an integral reservoir for a test fluid, and an actuator, so that the test fluid can be dispensed to facilitate the test in, for example, a lateral flow test strip. However, blood-based kits are relatively invasive as they require a needle prick to extract a sample of the patient's blood such that they are only suitable for use a clinical environment by a trained medical professional.

A need exists for a non-invasive and accurate approach for diagnosing the cause of sinusitis. Specifically, a need exists for a non-invasive and relatively accurate test for diagnosing in a patient presenting with symptoms of sinusitis the presence of a. There also exists a need for diagnosing other causes of sinusitis such as allergic rhinitis or upper respiratory tract viral infection.

A need exists for a method and/or apparatus and/or kit for diagnosing any one or more of bacterial sinus infection, upper respiratory tract viral infection or allergic rhinitis, that is relatively easy to use in a home or point of care setting and that provides results in minutes. It is desirable for any diagnostic apparatus, methods or kits for use in the home or in a point of care setting to be intuitive to use, that minimise opportunities for misuse, and provide reliable and accurate results in the hands of a non-experienced person. It is desirable to provide diagnostic apparatus, methods or kits that satisfy any one or more of the abovementioned needs to encourage their use.

U.S. Pat. No. 7,270,974 discloses a method and device for detecting and differentiating between allergic rhinitis, upper respiratory tract viral infection and bacterial sinusitis. The method and device includes a reagent strip upon which is fixed discrete indicators of pH, protein content, nitrite content, esterase activity, and eosinophil content of a sample with which said reagent test strip is contacted. Fresh secretion is collected on a non-absorbent surface and a reagent strip is immersed in the fresh secretion and removed immediately to avoid dissolution of reagents. A visual comparison of the reagents with a corresponding reagent chart is conducted to ascertain the results. The visual comparison process must occur at precise times after exposure of the reagents with the secretion. Specifically, the pH, protein and nitrite reading occurs at 60 seconds and the leukocyte reading occurs at 2 minutes. The pH and the protein readings can occur at any time up to 2 minutes after exposure.

However, the method and device of U.S. Pat. No. 7,270,974 is relatively difficult to use and is not optimised for accurate and repeatable results. The means of exposure of the sample to the reagent strip is relatively uncontrolled meaning that the reaction of the reagents to the sample may not be indicative of the characteristics of the sample. Furthermore, there are little effective controls in relation to the reading of the results such that use by an experienced user would be required for any kind of certainty in the results.

U.S. Pat. No. 9,606,118 discloses various devices, kits and assays for detecting pathogens associated with bacterial sinusitis. The specification discloses a rapid assay for the presence of one or more of the three most positive pathogens responsible for bacterial sinusitis, namely Haemophilus influenzae, Moraxella catarrhalis and Streptococcus pneumoniae. Various device embodiments are disclosed that include probes that are adapted to reach directly into the middle meatus region of the sinus and obtain a secretion sample directly. A collected secretion sample is analysed using a lateral flow assay and if the test is positive for one of the three common pathogens then an appropriate pharmaceutical treatment can be employed. If the result is negative then the patient may be treated for viral sinusitis and antibiotics may not be administered. The lateral flow assay may comprise a lysis buffer solution appropriate for use with all three pathogens and antigen binding agents specific to each type of bacteria. The binding agent acts as an indicator

However, the devices and methods of U.S. Pat. No. 9,606,118 are relatively invasive and requires insertion of a probe deep into the sinus cavity. It is unlikely that a patient will be willing the use such devices on themselves and instead it is likely the device would be used by a trained medical professional in a clinical setting only. Furthermore, the devices and methods of U.S. Pat. No. 9,606,118 are for the detection of bacterial infection only.

Any discussion of background art throughout the specification should in no way be considered as an admission that any of the documents or other material referred to was published, known or forms part of the common general knowledge.

### SUMMARY OF THE INVENTION

Accordingly, in one aspect, the invention provides a respiratory secretion sample collection device, including: a collection reservoir for directly receiving a sample of a respiratory secretion; a displacement member for insertion into the collection reservoir and displacing the sample within the collection reservoir; a container of a diluent for fluid communication with the sample for mixing the diluent with the sample; and an outlet for discharging the mixture of the diluent and the sample to an assay device.

Advantages of embodiments of the collection apparatus can include that they enable sanitary and non-invasive collection of respiratory secretions such as nasal mucus secretions. Other advantages of embodiments can include ensuring that all of the sample obtained within the collection apparatus is usable. Yet another advantage of embodiments can include the measurement of a precise and predetermined volume of the sample received from the patient. Still yet another advantage of embodiments can include enabling a predetermined volume of the sample to be mixed with a predetermined volume of diluent composition. By mixing precise volumes of the sample and the diluent the embodiments of the collection apparatus can enable the conduct of various assays for which the relative concentration of diluent and sample is critical. For example, assays for targeting biomarkers that are indicative of any one or more of a bacterial sinus infection, a viral respiratory infection, a fungal infection, an allergic immune response and an inflammatory response.

Throughout the specification the term “respiratory secretion” is used to refer to liquid samples taken from the nose and/or mouth including nasal and sinus mucus secretions, sinus aspirates or washouts/flushes, sputum, endotracheal or tracheal aspirates or secretions, nasopharyngeal aspirates or secretions. In addition, the term “respiratory secretion” includes samples taken from the nose or mouth that may include cerebrospinal fluid (CSF) in the event of a CSF leak. However, the term “respiratory secretion” is not intended to include other respiratory secretions such as saliva per se, urine, blood or sweat.

In embodiments, the displacement member comprises an elongated member including a resilient seal for sealing against an internal wall of the collection reservoir. Preferably, the resilient seal deforms upon insertion into the collection reservoir to provide a tight seal against the internal wall of the collection reservoir substantially throughout a complete stroke for displacing substantially all of the sample in the collection reservoir.

In embodiments, the collection device includes a cavity having a predetermined volume wherein insertion of the displacement member into the collection reservoir displaces the sample into the cavity for measuring out a predetermined volume of the sample.

In embodiments, the cavity is disposed within the displacement member and includes an inlet opening for fluid communication with the collection reservoir for the displaced sample to enter the cavity.

Preferably, the displacement member includes an internal space and the channel includes an outlet opening in fluid communication with the internal space for the sample to overflow into the internal space. Preferably, the channel includes a longitudinal passage extending between the inlet opening and the outlet opening. In embodiments, the outlet opening of the cavity is adapted for fluid communication with the container of the diluent.

In preferred embodiments, the container of the diluent includes an opening having a seal that is pierced by a protrusion adjacent to the outlet opening of the cavity by coupling the diluent container with the cavity.

In embodiments, the diluent container and the cavity are coupled together with a threaded connection or a snap fit connection.

In embodiments, the collection reservoir includes a void internal volume comprising a sample inlet opening and a resilient shroud adjacent to the sample inlet opening for providing a partial seal around a patient's nose.

The collection reservoir preferably includes an outlet opening for fluid communication with the cavity upon insertion of the displacement member into the reservoir.

The outlet opening of the collection reservoir includes a seal for directing the sample into the cavity by insertion of the displacement member into the collection reservoir. Preferably, the seal for the outlet opening of the collection reservoir is pierced by connection of the collection reservoir with a mixing chamber.

In embodiments, the mixing chamber includes an inlet opening and a protrusion adjacent to the inlet opening for piercing the seal of the outlet opening of the collection reservoir and for fluid communication between the container of diluent, the cavity and the mixing chamber.

The diluent container preferably includes a reciprocating plunger for displacing the sample in the cavity and the diluent between the diluent container and the mixing chamber for mixing the sample and the diluent.

In embodiments, the outlet for discharging the mixture of the diluent and the sample to an assay device includes an outlet of the mixing chamber to the assay device.

In embodiments, the diluent container includes graduated markings for controlling a volume of the diluent to be mixed with the sample and for controlling a volume of the mixture of the diluent and the sample discharged to the assay device.

Preferably, the collection reservoir includes a closed end for directing the sample into the cavity upon insertion of the displacement member into the reservoir.

In embodiments, the diluent container includes a mixing chamber in fluid communication with the cavity for mixing the sample and the diluent.

Preferably, the diluent container includes one or more baffles within the diluent container for mixing the sample and the diluent with a shaking action.

Preferably, the outlet for discharging the mixture of the diluent and the sample to an assay device includes an outlet of the diluent container to the assay device.

In embodiments, the assay device includes a lateral flow assay element within a housing adapted for insertion into the mixing camber and for displacing the mixture of the diluent and the sample into contact with the assay element within the housing.

Preferably, the housing and the mixing chamber are configured to displace a predetermined volume of the mixture into the housing upon insertion of the housing into the mixing chamber.

In another aspect, the invention provides an apparatus for the collection of a respiratory secretion sample and for the detection of any one or more of a bacterial infection, a viral infection, a fungal infection, an allergic immune response and an inflammatory response therefrom, the apparatus including: a respiratory secretion collection device for directly receiving a respiratory secretion sample; and a diagnostic element including one or more reagents.

Embodiments of the collection device include a reservoir contained within a housing including a nasal covering adapted to receive the nose and to direct a nasal secretion sample into the reservoir.

Preferably, insertion of an elongated member into the reservoir displaces the sample into a channel containing the diagnostic element.

In embodiments, the respiratory secretion collection device includes a protuberance for insertion into a nostril and the diagnostic element includes a recess that contains one or more reagents and that is shaped to receive and contact an exterior surface of the protuberance and thereby contact the reagents with the sample.

Embodiments include a lateral flow assay device configured for contacting the sample with the one or more reagents.

In another aspect, the invention provides a method for the collection of a respiratory secretion sample and for the detection of biomarkers indicative of any one or more of a viral respiratory infection, a bacterial sinus infection, a fungal infection, an allergic immune response and an inflammatory response, including:

collecting a respiratory secretion sample directly into a collection reservoir; displacing the sample within;
mixing the sample with a diluent;
discharging the mixture of the sample and the diluent to an assay device for determining the presence of biomarkers in the mixture indicative of any one or more of a viral respiratory infection, a bacterial sinus infection, a fungal infection, an allergic immune response and an inflammatory response.

In yet another aspect, the invention provides a kit for collecting and testing a respiratory secretion sample for the detection of biomarkers indicative of any one or more of a viral respiratory infection, a bacterial sinus infection, a fungal infection, an allergic immune response and an inflammatory response, including:

a collection reservoir for directly receiving a sample of a respiratory secretion;
a displacement member for insertion into the collection reservoir and displacing the sample within the collection reservoir;
a container of a diluent for fluid communication with the sample for mixing the diluent with the sample;
an outlet for discharging the mixture of the diluent and the sample to an assay device for determining the presence of biomarkers in the mixture indicative of any one or more of a viral respiratory infection, a bacterial sinus infection, a fungal infection, an allergic immune response and an inflammatory response.

In still yet another aspect, the invention provides an assay kit for detecting from a respiratory secretion sample any one or more of a viral respiratory infection, a bacterial sinus infection, a fungal infection, an allergic immune response and an inflammatory response, including:

a collection reservoir for directly receiving a sample of a respiratory secretion;
a displacement member for insertion into the collection reservoir and displacing the sample within the collection reservoir;
a container of a diluent for fluid communication with the sample for mixing the diluent with the sample;
an outlet for discharging the mixture;
an assay device for determining the presence of biomarkers in the mixture indicative of any one or more of a viral respiratory infection, a bacterial sinus infection, a fungal infection, an allergic immune response and an inflammatory response.

In another aspect, the disclosure herein provides an apparatus for the collection of a nasal secretion sample and for the detection of a bacterial sinus infection therefrom. The apparatus includes a nasal secretion collection device for collection of a nasal secretion sample and a diagnostic element including one or more reagents. The apparatus further includes a means for contacting the sample with the one or more reagents and for analysing the one or more reagents to determine if any one or more of a bacterial infection, a viral infection a fungal infection, an allergic immune response and an inflammatory response is indicated.

Various embodiments of the collection device are described and illustrated herein. Some embodiments include a collection device including a reservoir for receiving a respiratory secretion sample. The reservoir is contained within a housing. In some embodiments, the collection device includes a reservoir for receiving a respiratory secretion sample and a nasal covering adapted to receive the nose and to direct the sample into the reservoir. In embodiments, an elongated member supporting the reagents is provided for insertion into the reservoir to thereby contact the reagents with the sample.

In embodiments, insertion of the elongated member into the reservoir displaces the sample into a space or volume containing the diagnostic element. In an embodiment, the elongated member comprises a channel supporting the diagnostic element whereby insertion of the elongated member in the reservoir displaces the sample into the channel to thereby contact the sample with the diagnostic element.

Still further embodiments of the collection device are described and illustrated herein. Some embodiments include a collection device including a reservoir for receiving a nasal secretion sample. The reservoir is contained within a housing. A protuberance coupled to the housing for insertion into the nasal cavity is adapted to convey a nasal secretion from the nostril to the reservoir. The protuberance is preferably comprised of an absorbent material and in a preferred form has a base and an external surface converging towards an apex. Preferably, the external surface of the protuberance is stepped or is otherwise formed with a spiral step extending from the base to the apex. In another embodiment, the collection device includes a cylindrical housing or vial to which may be attached a flexible fitting for insertion into a nostril. In yet another embodiment, the collection device includes a card including absorbent sample strips or zones. In still yet another embodiment, the collection device may include a conical shaped absorbent member attached to a nasal irrigation syringe. In an embodiment, the collection device may include a sheet of flexible, absorbent material.

The diagnostic element preferably includes one or more reagents selected from the group including indicators of: pH; protein content; nitrite content; leukocyte esterase activity; and eosinophil content. Preferably, each of the reagents provides a visual indication that is indicative of a characteristic of the sample. The visual indication may be a change in colour, colour intensity or any visible indicator at all. In an embodiment, the indication is a binary positive or negative indication. In embodiments, the reagents may be configured to indicate a binary response, that is they may have one colour indicating a positive indication and another colour indicating a negative indication.

In embodiments, the diagnostic element or the assay may be comprised of a biosensor for the detection of one or more of the target chemical substances. In yet further embodiments, the diagnostic element or assay may comprise a DNA-based assay such as a polymerase chain reaction (PCR) assay to identify a target pathogen. In embodiments, the assay device includes a lateral flow assay device for determining the presence of biomarkers in the mixture indicative of any one or more of a viral respiratory infection, a bacterial sinus infection, a fungal infection, an allergic immune response and an inflammatory response.

The means for contacting the sample with the one or more reagents can include a component that contains or supports the reagents and maintains them in contact with the sample. An embodiment includes a recess that contains the reagents and that is shaped to receive and contact an exterior surface of a protuberance and thereby contact the reagents with the sample. The recess is preferably formed as part of a component that also includes a visual indicator such as an array of coloured indicator patches enabling visual comparison of the reagents with the indicator patches. In another embodiment, a lateral flow assay device is configured for contacting the sample with the one or more reagents. In yet another embodiment, an assay card is configured for receiving a sample strip containing the sample and the one or more reagents.

In another aspect, the invention provides a method for the collection of a nasal secretion sample and for the detection of a bacterial sinus infection therefrom. The method includes collecting a nasal secretion sample and contacting the sample with one or more reagents. The method further includes analysing the one or more reagents to determine if a bacterial infection is indicated. Various embodiments of the method are described herein.

Another aspect of the invention relates to a kit for collecting and testing a nasal secretion sample for the detection of any one or more of a bacterial sinus infection, a viral respiratory infection, a fungal infection, an allergic immune response and an inflammatory response therefrom. The kit includes a nasal secretion collection device for collection of a nasal secretion sample and embodiments thereof described herein. The kit further includes a diagnostic element including one or more reagents and embodiments thereof described herein. The kit further includes a means for contacting the sample with the one or more reagents and for analysing the one or more reagents to determine if a bacterial infection is indicated and embodiments thereof described herein. In yet another aspect, the invention provides an assay kit for detecting a bacterial sinus infection from nasal secretions.

### BRIEF DESCRIPTION OF THE FIGURES

The present invention will now be described in more detail with reference to preferred embodiments illustrated in the accompanying figures, wherein:

FIG. 1 illustrates a diagrammatic representation of a patient and a collection reservoir for directly receiving a sample of a nasal secretion;

FIG. 2 illustrates a perspective view of a nasal secretion sample collection device in accordance with an embodiment of the invention including the collection reservoir of FIG. 1 and a displacement member for insertion into the collection reservoir for displacing the sample into a channel of a predetermined volume for measuring out a volume of the sample, a diluent container including an opening for fluid communication with the channel for mixing the diluent with the volume of the sample, and an outlet for discharging the mixture of the diluent and the sample to an assay device;

FIG. 3 illustrates a partially exploded perspective view of the nasal secretion sample collection device of FIG. 2;

FIG. 4 illustrates a partially exploded frontal cross section view of the nasal secretion sample collection device of FIG. 2;

FIG. 5 illustrates a perspective view of the collection reservoir of the collection device of FIG. 2;

FIG. 6 illustrates a perspective view of the displacement member of FIG. 2;

FIG. 7 illustrates a frontal section view of the displacement member of FIG. 2;

FIG. 8 illustrates a perspective view of the diluent container of FIG. 2;

FIG. 9 illustrates a perspective view of a mixing chamber in accordance with an embodiment of the collection device of FIG. 2;

FIG. 10 illustrates a frontal section view of the mixing chamber of FIG. 9;

FIG. 11 illustrates the collection device of FIG. 2 and a microassay device;

FIG. 12 illustrates a diagrammatic representation of a patient and a collection reservoir for directly receiving a sample of a nasal secretion in accordance with another embodiment;

FIG. 13 illustrates a perspective view of a nasal secretion sample collection device in accordance with another embodiment of the invention including the collection reservoir of FIG. 12, a displacement member for insertion into the collection reservoir for displacing the sample into a channel of a predetermined volume, a diluent container including an opening for fluid communication with the channel for mixing the diluent with the volume of the sample, and an outlet for discharging the mixture of the diluent and the sample to an assay device;

FIG. 14 illustrates a partially exploded perspective view of the nasal secretion sample collection device of FIG. 13;

FIG. 15 illustrates a perspective view of the collection reservoir of the collection device of FIG. 13;

FIG. 16 illustrates a perspective view of the displacement member of FIG. 13;

FIG. 17 illustrates a perspective view of a frontal section view of the displacement member of FIG. 13;

FIG. 18 illustrates a perspective view of the diluent container of FIG. 13 in which internal components are represented in broken lines;

FIG. 19 illustrates a perspective view of the diluent container of FIG. 13 in which internal components are represented in broken lines and in which a volume of diluent composition is represented within the container;

FIG. 20 illustrates a diagrammatic representation of a patient and a device for the collection of a nasal secretion specimen in accordance with an embodiment of the invention;

FIG. 21 illustrates a perspective view of an apparatus for in vitro diagnosis of any one of more of a bacterial sinus infection, or a viral infection, a fungal infection, an allergic immune response and an inflammatory response including a nasal secretion collection device and an assay device in accordance with an embodiment of the invention;

FIG. 22 illustrates a perspective view of the nasal secretion collection device of FIG. 21;

FIG. 23 illustrates a perspective view of the apparatus of FIG. 21 supported in a vertical position;

FIG. 24 illustrates an end view of the apparatus of FIG. 21 illustrating reagent patches and an array of coloured indicators patches of the assay device;

FIG. 25 illustrates a perspective view of an apparatus for in vitro diagnosis of any one or more of a bacterial sinus infection, a viral infection, a fungal infection, an allergic immune response and an inflammatory response including a nasal secretion collection device in accordance with another embodiment of the invention;

FIG. 26 illustrates a perspective view of an assay device in for use with the nasal secretion collection device of FIG. 25;

FIG. 27 illustrates a front view of an apparatus for in vitro diagnosis of any one or more of a bacterial sinus infection, a viral infection, a fungal infection, an allergic immune response and an inflammatory response in accordance with another embodiment of the invention including a nasal secretion collection device, a reagent container, a pipette and an assay device;

FIG. 28 illustrates a front view of an apparatus for in vitro diagnosis of any one or more of a bacterial sinus infection, a viral infection, a fungal infection, an allergic immune response and an inflammatory response in accordance with another embodiment of the invention including a nasal secretion collection device and a container of a reagent;

FIG. 29 illustrates a front view of an assay card for use with the nasal secretion collection device and reagent of FIG. 28;

FIG. 30 illustrates a front view of the assay card of FIG. 29 in which a part of the nasal secretion collection device of FIG. 28 is fixed to the card and drops of the reagent are placed thereon;

FIG. 31 illustrates another embodiment of an apparatus for in vitro diagnosis of any one or more of a bacterial sinus infection, a viral infection, a fungal infection, an allergic immune response and an inflammatory response including reagent test patches on a flexible substrate.

FIG. 32 illustrates another embodiment of an apparatus for in vitro diagnosis of any one or more of a bacterial sinus infection, a viral infection, a fungal infection, an allergic immune response and an inflammatory response including a device for sinus irrigation and irrigated nasal secretion collection;

FIG. 33 illustrates another embodiment of an apparatus for in vitro diagnosis of any one or more of a bacterial sinus infection, a viral infection, a fungal infection, an allergic immune response and an inflammatory response including a device for sinus irrigation and irrigated nasal secretion collection;

FIG. 34 illustrates a perspective view another embodiment of an apparatus for in vitro diagnosis of any one or more of a bacterial sinus infection, a viral infection, a fungal infection, an allergic immune response and an inflammatory response including a nasal secretion collection device comprising mechanical vacuum apparatus and a tip comprising a hollow tube for insertion into the nostril wherein the assay is provided in the tip;

FIG. 35 illustrates a diagrammatic representation of a patient and the device of FIG. 3415 in use for collecting a nasal secretion specimen.

FIG. 36 illustrates a diagrammatic representation of a front view of an apparatus for in vitro diagnosis of any one or more of a bacterial sinus infection, a viral infection, a fungal infection, an allergic immune response and an inflammatory response including a nasal secretion collection device and an assay device in accordance with an embodiment of the invention;

FIG. 37 illustrates a diagrammatic representation of a perspective view of the apparatus of FIG. 36 including a housing having an elongated reservoir section and a funnel shaped section and an elongated stick for insertion into the reservoir section and supporting a diagnostic element;

FIG. 38 illustrates a view from another perspective of the elongated stick inserted within the housing;

FIG. 39 illustrates a reverse perspective view of the apparatus of FIG. 36;

FIG. 40 illustrates a side section view of the housing including the reservoir section and funnel section of FIG. 36;

FIG. 41 illustrates a top view of the housing of FIG. 36;

FIG. 42 illustrates a front view of the housing of FIG. 36;

FIG. 43 illustrates a side view of the elongated stick of the apparatus of FIG. 36;

FIG. 44 illustrates a front view of the elongated stick of the apparatus of FIG. 36;

FIG. 45 illustrates a top view of the elongated stick of the apparatus of FIG. 36;

FIG. 46 illustrates a perspective view of a nasal secretion sample collection device in accordance with another embodiment of the invention wherein the diluent container not only serves the function of the mixing chamber but an assay device housed within a plunger is adapted for insertion into the diluent container;

FIG. 47 illustrates a front view of the collection device of FIG. 46;

FIG. 48 illustrates a perspective view of the collection device of FIG. 46, wherein some components are represented in broken lines to reveal the relative location of other components therein, and wherein the plunger housing the assay device is aligned with the diluent container for insertion therein;

FIG. 49 illustrates a reverse perspective view of the device of FIG. 46 wherein some components are represented in broken lines to reveal the relative location of other components therein, and wherein the plunger housing the assay device is aligned with the diluent container for insertion therein;

FIG. 50 illustrates a perspective view of the plunger housing the assay device, wherein a wall surrounding a hollow internal cavity containing the lateral flow assay strip is partially cut away to reveal the lateral flow assay strip located therewithin; and

FIG. 51 illustrates a front view of the plunger housing the assay device wherein a test zone of the lateral flow assay strip including one of more visual indicators thereof is visible through a window in the wall of the housing 984.

The invention will now be described in further detail with reference to the embodiments illustrated in the Figures.

### DETAILED DESCRIPTION

The present invention broadly relates to apparatus and methods for the collection of a respiratory secretion sample. In embodiments, the collection apparatus is adapted to discharge the sample to a diagnostic device, such as a microassay chip device or a lateral flow assay device. In other embodiments, the collection apparatus is part of a collection and diagnostic device including one or more reagents for biomarker detection. Embodiments of the collection apparatus are adapted for diagnosing from the sample a cause of sinusitis including any one or more of a bacterial sinus infection, a viral infection, a fungal infection, an allergic immune response and an inflammatory response.

Several embodiments of the apparatus are disclosed herein. However, the apparatus broadly includes a reservoir for receiving respiratory secretions such as nasal mucus secretions and/or sputum.

In some embodiments, the apparatus includes one or more reagents adapted for indicating the presence of one or more biomarkers in the sample and a means for analysing the one or more reagents to determine if a any one or more of a bacterial sinus infection, a viral infection, a fungal infection, an allergic immune response and an inflammatory response is indicated. In embodiments, the apparatus includes one or more reagents adapted for indicating the presence of Cerebrospinal fluid (CSF) in the nasal mucus or sputum sample.

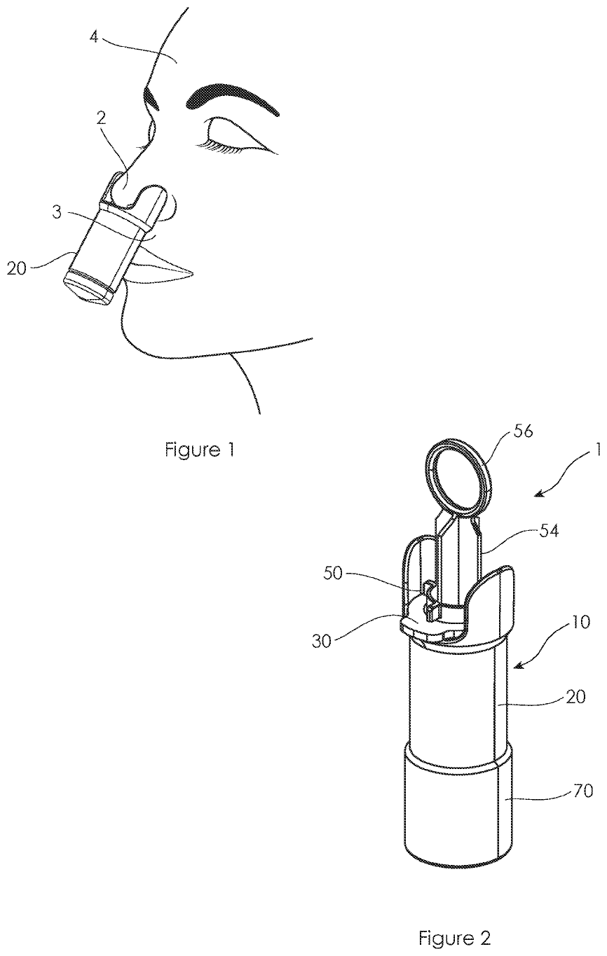

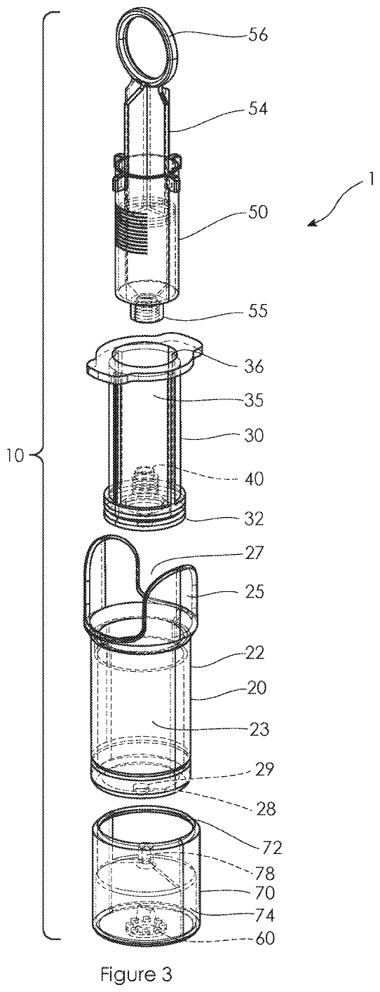

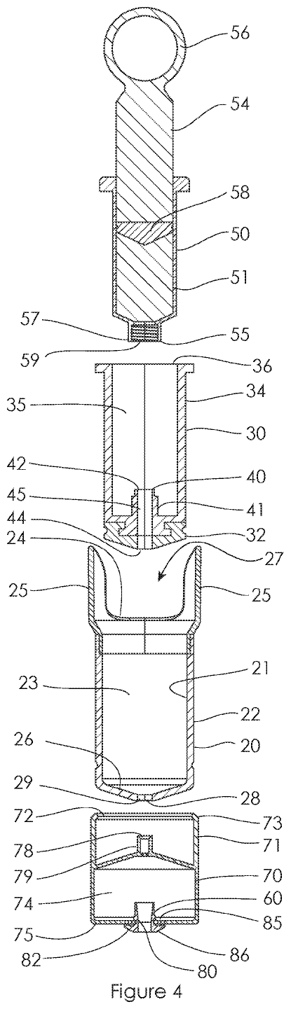

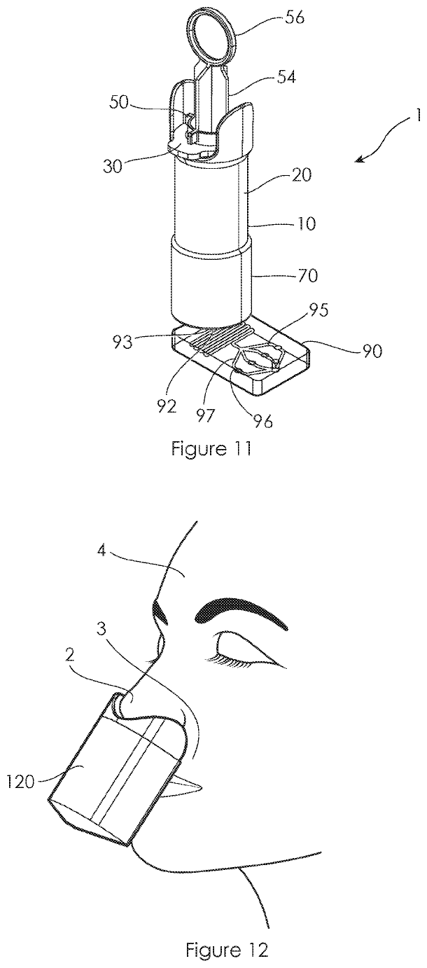

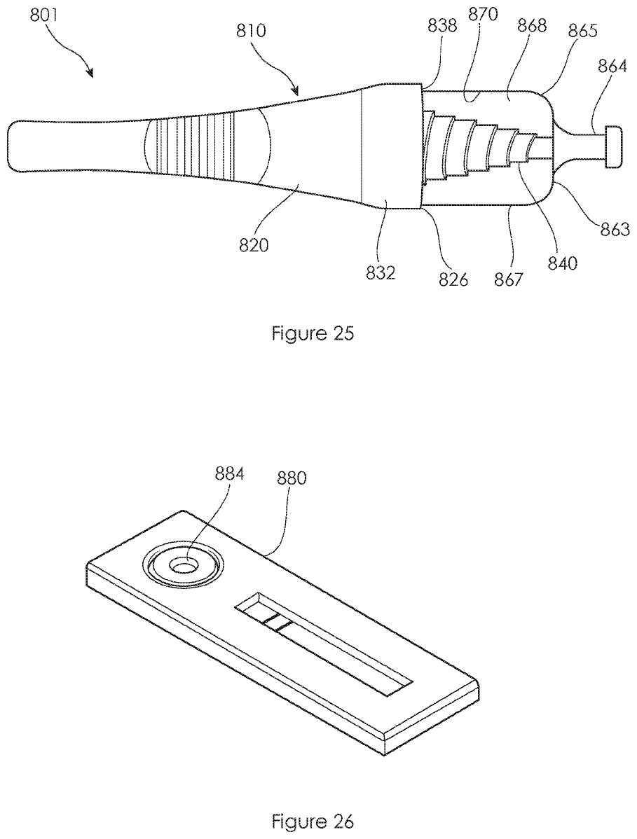

FIGS. 1 to 11 illustrate a diagrammatic representation of an apparatus 1 for the collection of a respiratory secretion in accordance with an embodiment of the invention. The apparatus 1 includes a respiratory secretion collection device 10 including a collection reservoir 20 for directly receiving a sample of a respiratory secretion. The device 10 also includes a displacement member 30 for insertion into the collection reservoir 20 and displacing the sample within the collection reservoir 20. The device 10 also includes a container 50 of a diluent for fluid communication with the sample for mixing the diluent with the sample. The device 10 further includes an outlet 60 for discharging the mixture of the diluent and the sample to an assay device, such as a microfluidic chip based assay device 90 illustrated in FIG. 11 or a lateral flow assay device such as the lateral flow assay device 880 of FIGS. 25 and 26. The collection reservoir 20 is adapted for the collection of nasal secretions or sputum.

Referring to FIGS. 1 to 9, the collection reservoir 20 includes a barrel 22 comprising an internal wall 21 defining a longitudinal internal void volume 23 therewithin. The internal wall 21 is cylindrical, but it may be formed with an oval or another suitably shaped profile. The barrel 22 includes a sample inlet opening 24 at one end and a tapering wall 26 at an opposite end. A resilient shroud comprised of resilient wing members 25 are disposed adjacent to the sample inlet opening 24. A pair of opposite recesses 27 are defined between the wing members 25. As illustrated in FIG. 1, in use, the opposite recesses 27 are for receiving the bridge or tip of the nose 2 and the top lip 3 of a patient 4. In use, one wing member 25 can be pressed against one nostril to close the nostril while the other nostril remains open when the patient blows their nose into the collection reservoir 20. The other wing member 25 provides a cover to prevent nasal mucus from spraying uncontrollably.

In use, the resilient shroud comprised of the wing members 25 and the recesses 27 provide at least a partial seal around the nose 2 or nostrils of the patient 4. The patient blows their nose 2 and nasal secretions are collected in the void volume 23. The tapering wall 26 of the collection reservoir 20 includes a central outlet opening 28. The outlet opening 28 is closed by a foil seal 29 that is secured with an adhesive to the wall 26 surrounding the opening 28. The seal 29 maintains the sample within the void volume 23 when the sample is being collected from the patient.

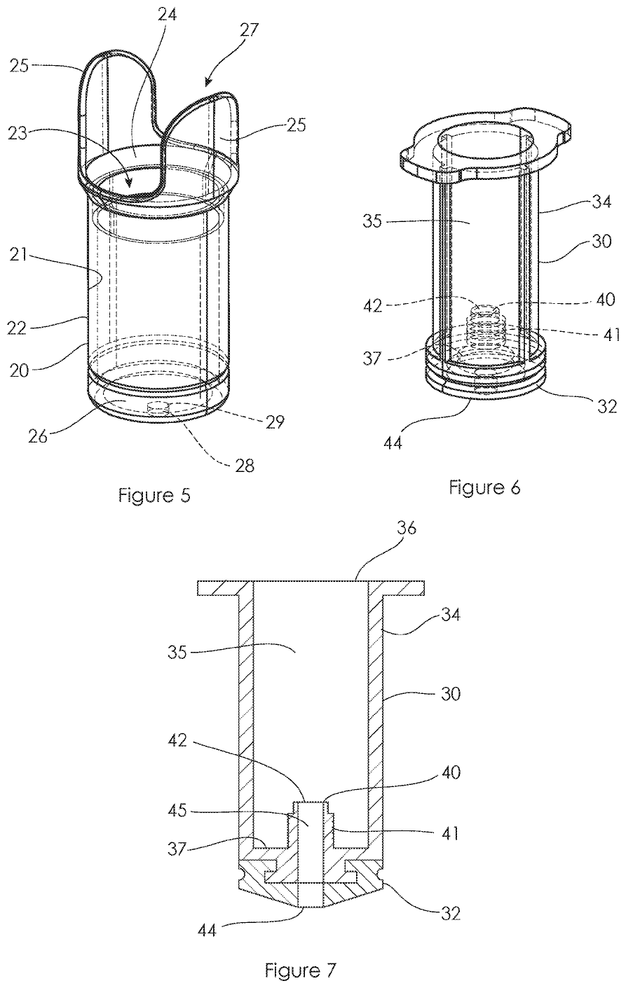

Referring to FIGS. 6 and 7, the displacement member 30 comprises an elongated member comprised of a hollow shaft 34 comprising an internal space 35. The space 35 includes an opening 36 at one end and a wall 37 at an opposite end. The displacement member 30 includes a cavity 40 of a predetermined volume. The cavity 40 is located centrally within the wall 37 and includes a cylindrical member 41 including a first opening 42, a second opening 44 and a longitudinal passage 45 extending therebetween. The first opening 42 is within the wall 37 at the distal end of the plunger 30. The hollow shaft 34 of the displacement member 30 includes a resilient seal 32 at the distal end thereof that is configured for sealing against the internal wall 21 of the collection reservoir 20.

Upon insertion of the displacement member 30 into the collection reservoir 20, the first opening 42 of the cavity 40 is in fluid communication with the void volume 23 containing the sample deposited by the patient. The displacement member 30 displaces the sample towards the tapering wall 26 at the distal end of the chamber 23 and into the cavity 40 via the first opening 42. The resilient seal 32 deforms upon insertion into the collection reservoir 20 to provide a tight seal against the wall 21 of the reservoir 20 substantially throughout a complete stroke. The resilient seal 32 ensures that substantially all of the sample within the chamber 23, including any sample on the wall 21, is wiped down the wall 21 and is displaced throughout the entire stroke of the plunger 30.

The viscosity of respiratory secretion samples, such as nasal secretions, can vary from between about 500 to about 25,000 centipoise (cP). Low viscosity samples are typically between about 500 to 5,000 or 10,000 cP and high viscosity samples are between about 5,000 or 10,000 and 25,000 cP. An advantage of embodiments in which the displacement member 30 includes the resilient seal 32 is that with relatively low viscosity samples and relatively high viscosity samples, substantially all of the sample within the chamber 23 is displaced into the cavity 40 and is usable.

The sample that is displaced into the longitudinal passage 45 of the cavity 40 fills the volume therewithin and any overflow passes out of the second opening 44 and into the hollow internal space 35 within the hollow shaft 34. Because the internal volume of the longitudinal passage 45 is predetermined, a known volume of the sample is measured out and obtained within the cavity 40.

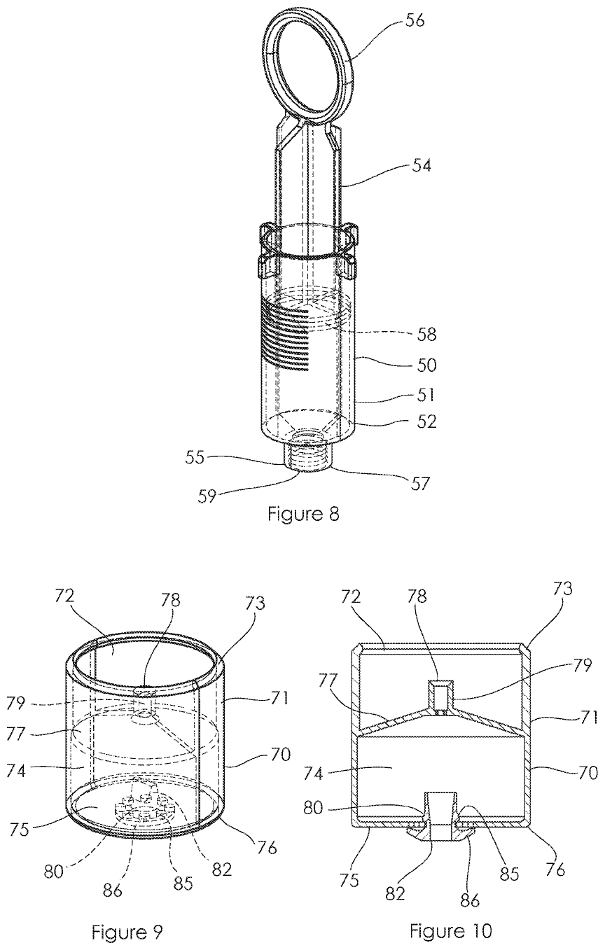

Referring to FIG. 8, the diluent container 50 is a syringe 52 comprising a hollow tube 51 prefilled with a liquid diluent composition and a plunger 53. In the embodiment illustrated in FIGS. 1 to 9, the plunger 53 includes a piston 58 coupled to one end of an elongated rod 54 and a ring grip 56 coupled to the other end of the rod 54. The tube 51 includes a hollow cylindrical tip 57 at a distal end thereof. An outlet opening 55 of the diluent container 50 is located at an end of the cylindrical tip 57. The outlet opening 55 is closed by a foil seal 59 that is secured with an adhesive to the cylindrical tip 57 surrounding the opening 28. Preferably, the volume of the diluent within the diluent container 50 is at a ratio of 5 to 1 the volume of the sample in the cavity 40. Alternatively, the ratio of the volume of the diluent to the volume of the cavity 40 can be any one of a ratio of about 1:1, 2:1, 3:1, 4:1, 6:1, 7:1, 8:1, 9:1 or 10:1.

The cylindrical tip 57 of the syringe 52 is configured for connection at the second end 44 of the cylindrical member 41 by way of a threaded connection, although a snap fit connection may be used instead. Upon connection of the tip 57 with the tubular member 41 the seal 59 closing the outlet opening 55 is pierced by the tubular member 41 surrounding the cavity 40. Fluid communication is thereby established between the hollow tube 51 containing the diluent composition and the cavity 40 containing a predetermined volume of the sample.

Referring in particular to FIGS. 9 and 10, the apparatus 1 further includes a mixing chamber 70 adapted for connection to the outlet opening 28 of the collection reservoir 20. The mixing chamber 70 includes a hollow cylindrical body 71 including an opening 72 at a proximal end 73 for receiving a distal end of the barrel 22 of the collection reservoir 20 in a snap fit or an interference fit or a threaded coupling. The mixing chamber 70 has an end wall 75 at a distal end 76 of the mixing chamber 70 and an intermediate wall 77 between the proximal end 73 and the distal end 76 of the cylindrical body 71. A mixing zone 74 is defined in a space between the intermediate wall 77 and the end wall 75. The intermediate wall 77 includes a central opening 78 surrounded by a cylindrical protrusion 79 upstanding from the intermediate wall 77.

Upon connection of the distal end of the barrel 22 of the collection reservoir 20 within the opening 71 at the proximal end 73 of the mixing chamber 70, the seal 29 closing the outlet opening 28 of the collection reservoir 20 is pierced by the cylindrical protrusion 79. Fluid communication is thereby established between the central opening 78 in the intermediate wall 77 of the mixing chamber 70 and the outlet opening 28 of the collection reservoir 20.

Thus, the predetermined volume of the sample and the diluent composition can enter the mixing zone 74.

The end wall 75 of the mixing chamber 70 includes a central opening 80 surrounded by a ring of smaller apertures 85. The central opening 80 includes a one way valve 82, in the form of a duck-bill valve, that is configured to allow air to enter the mixing zone 74 but to prevent liquid from exiting the mixing zone 74. The ring of smaller apertures 85 include a one-way valve 86 in the form of an umbrella valve that allows air to exit the mixing zone 74 and to close the apertures 85.

The diluent composition is mixed with the predetermined volume of the sample by gripping the ring grip 56 of the diluent container 50 and axially translating the piston 53, the rod 54 and the ring grip 56 in an out of the tube 51. Axial translation of the piston 53 in one direction causes the diluent composition and the predetermined volume of the sample to be displaced into the mixing zone 74. Air in the mixing zone 74 displaced by the mixture of the diluent and the sample can escape through the umbrella valve 86. Axial translation of the piston 53 in the opposite direction causes the diluent composition and the predetermined volume of the sample to be drawn out of the mixing zone 74 and into the diluent container 50. Air can be drawn into the mixing zone 74 through the duck bill valve 82 to equalise the pressure within the mixing zone 74 and the external air pressure. Reciprocal translation of the piston 53 in and out of the tube 51 causes the diluent and sample mixture to move into and out of the mixing zone 74 to thereby thoroughly mix the diluent and the sample.

The method of mixing the sample and the diluent described above comprises a form of turbulent mixing. Such turbulent mixing can be achieved by other means not illustrated in the drawings, including turbulent mixing by a helical or screw member or with vortices. In other embodiments, mixing is achieved by stirring the mixture mechanically, magnetically, electrically or acoustically.

In the embodiment of FIGS. 1 to 9, the central opening 80 of the mixing chamber 70 serves as the outlet 60 for discharging the mixture of the diluent and the sample to the assay device 90, as illustrated in FIG. 11. The tube 51 of the diluent container 50 is formed out of a transparent polymer material and includes graduated markings. The graduated markings can be employed for controlling a volume of the diluent to be mixed with the sample and in some embodiments for controlling a volume of the mixture of the diluent and the sample that is discharged to the assay device 90.

The embodiment of the assay device 90 illustrated in FIG. 11 is a microassay device 95 that includes a plurality of reaction chambers 96 for exposing the diluent-sample mixture with one or more reagents for biomarker detection. The assay device 90 includes an inlet port 92 that is adapted to mate with the central opening 80 of the mixing chamber 70. The inlet port 92 includes a protruding pipe member 93 that is adapted to be inserted into the duck-bill valve 82 to establish fluid communication between the mixing zone 74 and the inlet port 92 of the microassay device 95. Movement of the piston 53 axially into the tube 51 of the diluent container 50 displaces a volume of the diluent-sample which is injected into the microassay device 95 via the inlet port 92.

The microassay device 95 includes a plurality of conduits 97 that convey the diluent-sample mixture to the reaction chambers 96. Analytes within the diluent-sample mixture react with the reagents within the reaction chambers 96. The occurrence of such reactions indicates the presence of target analytes in the diluent-sample mixture such as analytes that are indicative of any one or more of a bacterial sinus infection, a viral respiratory infection, a fungal infection, an allergic immune response and an inflammatory response.

The microassay device 95 can include a biological component and a physicochemical detector. The biological component may include one or more enzymes or antibodies to target an analyte such as any one or more of those described herein. The transducer or detector is adapted for detecting a reaction between the target analyte and the biological component. The transducer may be comprised of an optical, piezoelectric, electrochemical or an electro-chemiluminescence detector.

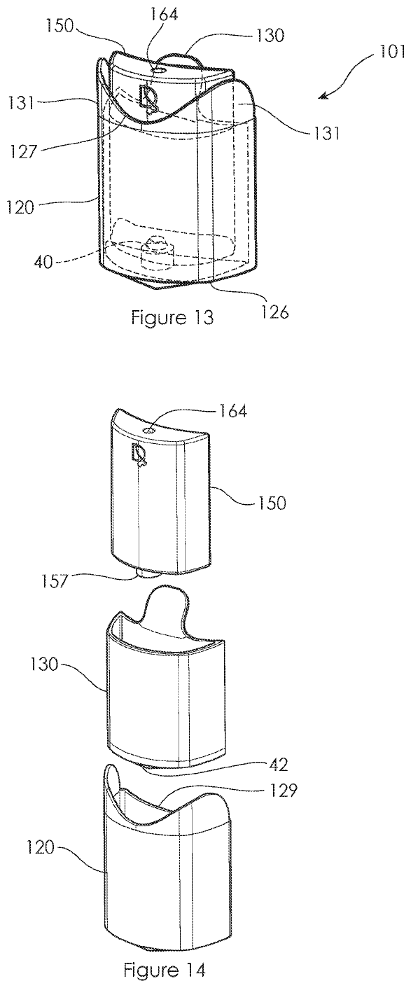

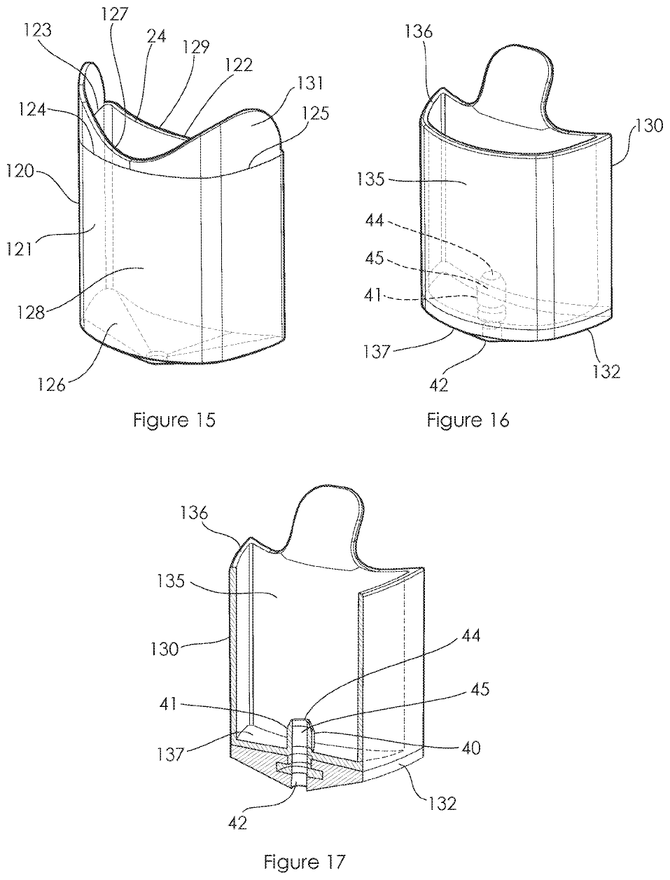

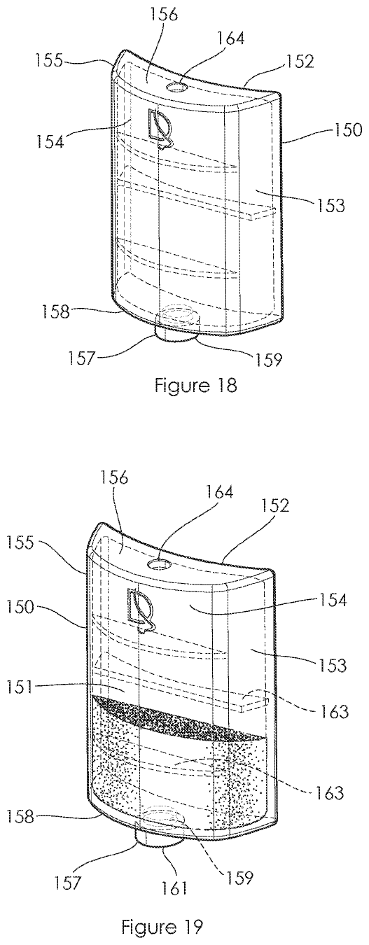

Referring to FIGS. 12 to 19, another embodiment of the invention comprising an apparatus 101 for the collection of a respiratory secretion is illustrated. The embodiment of the apparatus 101 of FIGS. 11 to 17 includes many functionally similar features to the embodiment of FIGS. 1 to 10 and as such like reference numerals will be used to identify like features.

The embodiment of the apparatus 101 of FIGS. 12 to 19 differs in that the diluent container 150 also serves the function of the mixing chamber 70 of the embodiment of FIGS. 1 to 10. Another difference is that the diluent container 150 includes an internal chamber of fixed volume and does not include a means for changing an internal volume of the diluent container 150 as the plunger 53 does in the syringe 52 of FIGS. 1 to 10.

Referring to FIGS. 12 to 19, the collection reservoir 120 has an opening 24 at one end and a closed, tapering wall 26 at an opposite end. The opening 24 is defined by a rim 129 that includes a pair of opposite, curved rim sections 122, 124 and a pair of opposite, converging and substantially linear lateral rim sections 123, 125 extending between the curved rim sections 122, 124. One of the curved rim sections 122 is adapted to conform with the shape of a patient's upper lip. The collection reservoir 120 includes an internal wall 121 defining a longitudinal internal chamber 128 therewithin. The internal wall 121 has the same profile as the rim 129 throughout its length to the tapering wall 126.

Resilient wing members 131 are disposed on the linear rim sections 123, 125. As illustrated in FIG. 12 a recess 127 is defined between the wing members 125 for receiving the bridge of the nose of a patient. In use, the wing members 125, the recess 127 and one of the curved rims sections 122 form a shroud that provides a partial seal around the nose of a patient. The patient blows their nose 2 and nasal secretions are collected in the void volume 23. The tapering wall 126 at the opposite end of the void volume 23 is sealed and has no central opening.

As illustrated in FIGS. 16 and 17 the displacement member 130 includes a hollow internal space 135 with an opening 136 at one end and a wall 137 at an opposite end. The cavity 40 is located centrally within the end wall 137. Like the previous embodiment, the first opening 42 is located within the end wall 137 at the distal end of the displacement member 30. The displacement member 130 includes a resilient seal 132 at the distal end thereof that is configured for tightly sealing against the internal wall 121 of the collection reservoir 120.

Upon insertion of the displacement member 130 into the collection reservoir 120, the first opening 42 of the cavity 40 is in fluid communication with the void volume 119 containing the sample deposited by the patient. The displacement member 130 displaces the sample towards the tapering wall 126 at the distal end of the void volume 119 and into the cavity 40 via the first opening 42. The resilient seal 132 deforms upon insertion into the collection reservoir 120 to provide a liquid tight seal against the wall 121 of the reservoir 120 substantially throughout a complete stroke. The resilient seal 132 ensures that all of the sample within the void volume 119, including any sample on the wall 121, is displaced throughout the entire stroke of the displacement member 130.

The sample displaced into the longitudinal passage 45 of the cavity 40 fills the volume therewithin and any overflow passes out of the second opening 44 and into the hollow internal space 135 within the displacement member 130. Because the internal volume of the longitudinal passage 45 is predetermined, a known volume of the sample is obtained within the cavity 40.

An advantage of embodiments in which the displacement member 130 includes the resilient seal 132 is that with relatively low viscosity samples and relatively high viscosity samples, substantially all of the sample within the void volume 119 is displaced into the cavity 40 and is usable.

The diluent container 150 comprises an internal chamber 151 prefilled with a liquid diluent composition. The internal chamber 151 is defined by an internal wall 156 that includes a pair of opposite, curved wall sections 152, 154 and a pair of opposite, converging and substantially linear side wall sections 153, 155 and a pair of opposite end walls 156, 158. A distal one of the end walls 158 includes a protruding, hollow cylindrical tip 157 which includes an opening 159 that is closed by a foil seal 161 secured with an adhesive to the cylindrical tip 157. The cylindrical tip 157 is configured for connection to the second end 44 of the cylindrical member 41 by way of a snap fit connection. Connection of the tip 157 with the cylindrical member 41 pierces the seal 161 closing the opening 159. Fluid communication is thereby established between the chamber 151 containing the diluent composition and the cavity 40 containing a predetermined volume of the sample.

Shaking, or simply inverting, the assembled diluent container 150, plunger 130 and collection reservoir 120 mixes the diluent and the volume of the sample in the internal chamber 151 of the diluent container 150. The diluent container 150 includes a series of internal baffles 163 fixed to the side wall sections 152, 153, 154, 155 within the internal chamber 151 to enhance mixing achieved by shaking the container 150.

The proximal end wall 156 defining the internal chamber 151 of the diluent container 150 includes a central aperture 164. The central aperture 164 serves as the outlet 60 for discharging the mixture of the diluent and the sample to the assay device 90, such as the exemplary microassay device 95 of FIG. 10. The central aperture 164 is closed by a foil seal 169 that is secured with an adhesive to the wall 156 surrounding the aperture 164. In use, the seal 169 is adapted to be pierced, such as upon mating with the inlet port 92 of the microassay device 95 of FIG. 10.

Advantages of the embodiments of the collection apparatus 1, 101 of FIGS. 1 to 11 and 12 to 19, include that they can enable the relatively convenient and non-invasive collection of a nasal secretion sample from a patient. Other advantages can include that the collection apparatus 1, 101 ensures that all of the sample obtained from the patient into the collection reservoir 20, 120 is usable. Other advantages of embodiments of the collection apparatus 1, 101 can include measuring a precise and predetermined volume of the sample received from the patient and mixing with a predetermined volume of the diluent composition. By mixing precise volumes of the sample and the diluent the embodiments of the collection apparatus 1, 101 of FIGS. 1 to 11 and 12 to 19 facilitate the conduct of various assays for which the relative concentration of diluent and sample is critical. For example, assays for targeting biomarkers that are indicative of any one or more of a bacterial sinus infection, a viral infection, a fungal infection, an allergic immune response and an inflammatory response.

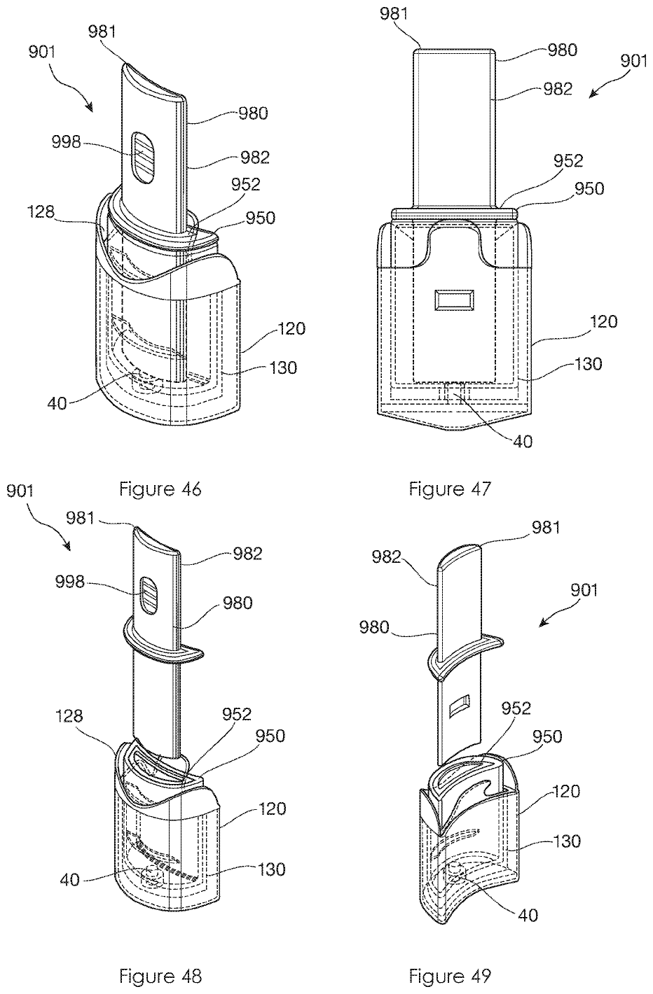

FIGS. 46 to 51 illustrate another embodiment of an apparatus 901 for the collection of a respiratory secretion. The embodiment of the apparatus 901 of FIGS. 46 to 51 includes many functionally similar features to the embodiment of FIGS. 12 to 19 and as such like reference numerals will be used to identify like features.

The embodiment of the apparatus 901 of FIGS. 46 to 51 differs in that the diluent container 950 not only serves the function of the mixing chamber but an assay device 980 housed within a plunger 982 is adapted for insertion into the diluent container 950. In other respects, the apparatus 901 is similar to the embodiment of the apparatus 101 of FIGS. 12 to 19.

The apparatus 901 includes the collection reservoir 120 including the internal void volume 128 for receiving the sample. The displacement member 130 including the hollow internal space 135 and the resilient seal 132 is insertable into the collection reservoir 120 to displace the sample into the cavity 40 within the displacement member 130. The sample fills the cavity 45 and any overflow passes out into the hollow internal space 135. The diluent container 950 is inserted into the displacement member 130 which brings the internal chamber 951 of the diluent container 950 into fluid communication with the sample within the cavity 40. The diluent and the sample are mixed within the internal chamber 951 of the diluent container by, for example, shaking the apparatus 901.

As illustrated in FIGS. 46 to 49, the diluent container 950 has an opening 952 at the proximal end that is sealed, for example by a foil seal. The opening 952 is adapted to receive the assay device 980 therewithin as illustrated in FIGS. 46 and 47. Insertion of the assay device 980 pierces the foil seal covering the opening 952.

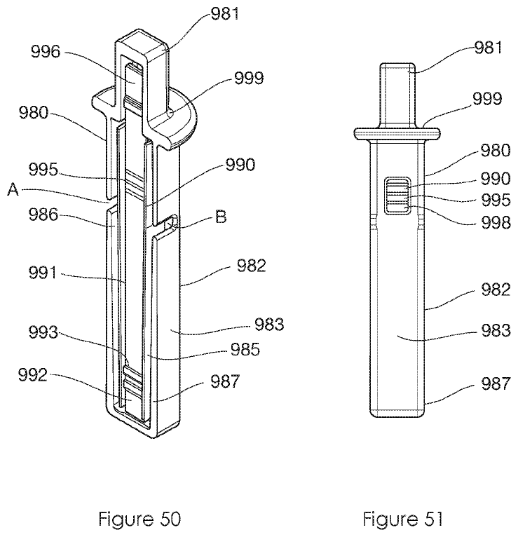

Referring to FIGS. 50 and 51, the assay device 980 includes a lateral flow assay 990 housed within an elongated plunger 982. The lateral flow assay 990 includes an elongated backing 991, a sample pad 992 at one end for contacting the mixture of the diluent and the sample. Next to the sample pad is a conjugate zone 993 and a test zone 995. The mixture is wicked through the backing from the sample pad 992, through the conjugate zone 993 comprising labelled tags combined with biorecognition elements and then onto the test zone 995 for target DNA-probe DNA hybridization or antigen-antibody interaction. An absorbent zone 996 is located at an end of the backing 991 opposite to the sample pad 991 for reserving waste.

The elongated plunger 982 includes a shaft 983 including a hollow internal cavity 985 containing the lateral flow assay 990. The hollow internal cavity 985 is surrounded by an enclosing wall 986 that includes a pair of openings A, B that are located at a predetermined position along the length of the shaft 983. The sample pad 992 of the lateral flow assay 990 is located at a distal end 987 within the hollow cavity 985. The test zone 995 including one of more visual indicators 997 thereof are visible through a window 998 through the wall 986 of the housing 984 located towards a proximal end 999. The plunger 982 includes a grip section 981 at the proximal end 999.

In use, the plunger 982 is inserted into the internal chamber 951 of the diluent container 950, distal end 986 first. The external dimensions of the shaft 984 of the plunger 982 are configured to displace a precise amount of the mixture of the sample and the diluent within the internal chamber 951 of the diluent container 950 so that a predetermined volume of the mixture enters the cavity 985 through the openings A, B. Accordingly, the external dimensions of the shaft 984, the internal dimensions of the internal chamber 951 and the volume of the sample and diluent mixture and the locations of the openings A, B along the length of the shaft 984 are factors that together determine the volume of the mixture that enters the cavity 985. The desired volume of the mixture that enters the cavity 985 may be 1 mL, 2 mL, 3 mL, 4 mL, 5 mL or between about 0.2 to 4.0 mL or any amount therebetween. The desired volume is determined so that the mixture contacts only the sample pad 992 of the of the lateral flow assay 990.

The lateral flow assay 990 housed within the plunger 982 is configured for targeting biomarkers that are indicative of any one or more of a bacterial sinus infection, a viral infection, a fungal infection, an allergic immune response and an inflammatory response.

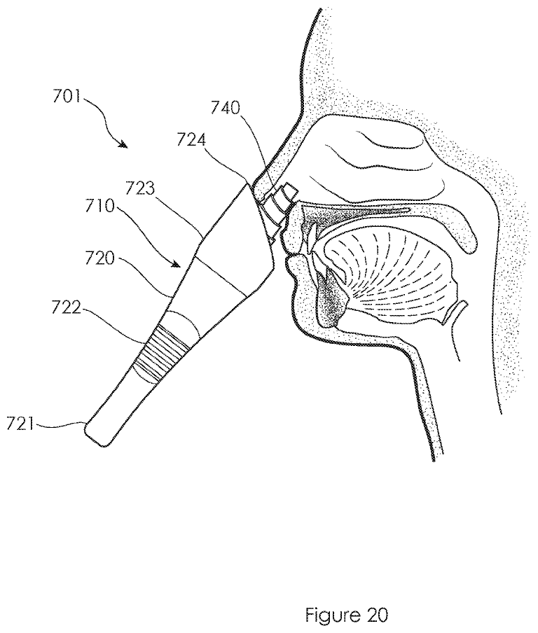

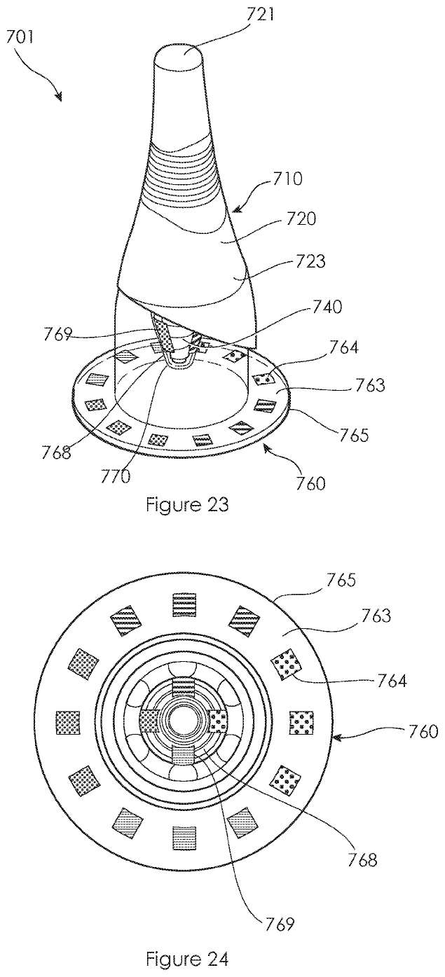

FIGS. 20 to 24 illustrate a diagrammatic representation of an apparatus 701 for the collection of a nasal secretion sample and for the diagnosis of any one or more of a bacterial sinus infection, a viral infection, a fungal infection, an allergic immune response and an inflammatory response from the sample. The apparatus includes a device 710 for the collection of a nasal secretion specimen in accordance with an embodiment of the invention. The device 710 includes a housing 720 having an elongated funnel shaped form extending from a closed end 721 to an open end 724. The closed end 721 has a smaller diameter than the open end 724 and an external surface 723 of the housing tapers from the open end 724 to the closed end 721.

The open end 724 of the housing 720 includes an aperture 726 that opens to a recess 730. The recess 730 is defined by a base 734 and a peripheral wall 732 upstanding from a periphery of the base 734 and terminating at an upper edge 738 that defines the aperture 726. The upper edge 738 of the peripheral wall 732 is shaped to provide a chamfered or sloping profile. Towards the closed end 721, the housing 720 contains a hollow internal cavity that functions as a reservoir 725 for receiving nasal secretions. The base 734 separates the recess 730 from the reservoir 725.

The device 710 includes a protuberance 740 for insertion into a nostril. The protuberance 740 upstands from the centre of the base 730. The protuberance 740 has a bottom surface 742 and an external surface 45 converging towards an apex 747. The external surface 745 is formed with a series of steps 746. In the embodiment illustrated in FIGS. 20 to 245, a continuous spiral step 746 is formed in the external surface 745 and extends from the bottom 742 to the apex 747.

In use, the protuberance 740 is inserted into a nostril as illustrated in FIG. 1 and the steps 746 are provided to collect the nasal secretion. A portion of the external surface 723 towards the closed end 721 functions as a handle 722 shaped to be gripped by a user for insertion into the nostril as illustrated in FIG. 1.

The protuberance 740 is formed out of a flexible material and preferably also an absorbent material such as a sponge. In an embodiment, the material from which the protuberance is formed is a cellulose acetate sponge. This material can help to stimulate nasal mucus and absorb it. However, other materials may be employed that are also capable of stimulating the secretion of nasal mucus and/or absorbing the nasal mucus. The protuberance 740 is adapted to deform to the shape of the nostril upon insertion and preferably to absorb some of the specimen. The spiral form the step 746 allows the specimen to travel down towards the bottom of the protuberance 740.

The upper edge 738 of the peripheral wall 732 is adapted for contact with the face when the protuberance 740 is inserted into the nostril. Accordingly, the wall 732 functions as a shroud to prevent nasal secretions from being spread uncontrollably.

The base 734 has a plurality of openings 736 that extend through the base 734 between the recess 730 and the reservoir 725. The openings 736 are in the base 730 about the protuberance 740 and are adapted to allow nasal secretions to drain from the protuberance 740 and/or the recess 730 into the reservoir 725.

The apparatus 701 further includes a diagnostic element 760 comprising a cap 765 adapted for connection to the aperture 726 of the housing 720. The cap 765 is comprised of a flat disc shaped base plate 763 and a cylindrical body 767 upstanding from the base plate 763. The cap 765 and the cylindrical body 767 are preferably formed out of a clear plastic material. The base plate 763 includes a circular array of coloured patches 764 surrounding the cylindrical body 767. The cylindrical body 767 has an internal conical shaped recess 768 defined by a conically shaped surface 770 with reagent patches or strips 769 provided thereon.

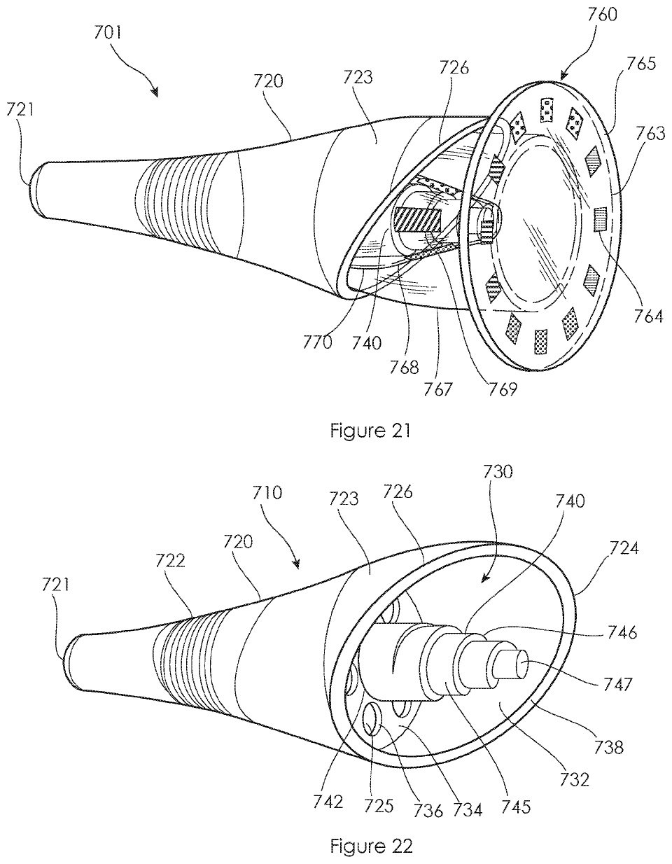

As illustrated in FIGS. 21, 22 and 23, the cylindrical body 767 is adapted for connection with the aperture 726 of the housing 720. The cylindrical body 767 may be sized and shaped for an interference fit or snap fit with the wall 732 of the housing 720. The conical recess 768 receives the protuberance 740 therewithin whereby the reagent patches or strips 769 meet or come into contact with the external surface 745 of the protuberance 740.

Once the cap 765 is fitted to the collection device 710 the entire assembly is stood upright on the disc shaped base plate 763 and left fora period. Under the action of gravity, the nasal secretions in the reservoir 725 pass back through the apertures 726 in the base 734 and into the conical recess 768 of the cap 765. Accordingly, nasal secretions absorbed in the protuberance 740 and/or that pass from the reservoir 725 into the conical recess 768 are exposed to the reagent patches or strips 769 on the internal conical surface 770 of the cap 765.

The one or more reagents are selected from the group including indicators of pH, protein content, nitrite content, leukocyte esterase activity, and eosinophil content. Such reagent strips may include those that are commonly employed for testing urine, or ‘urine dipsticks’, to diagnose UTI, diabetes and kidney disorders. Exemplary reagents include: pH: methyl red and bromothymol blue; Protein: tetrabromophenol blue; Nitrite: p-arsanilic acid; 1,2,3,4-tetrahydrobenzo-(h)-quinolin-3ol; Leukocytes: derivatized pyrrole amino acid ester; diazonium salt.

As illustrated in FIGS. 21, 23 and 24, and as described above, the cap 765 is formed out of transparent plastic to allow light to reach the reagent patches or strips 769. When a period of time has elapsed for the reagents to react with the nasal secretions, as illustrated in FIG. 24, the entire assembly can be tilted to allow viewing of the circular array of coloured patches 764 and the reagent patches or strips 769 through the transparent base plate 763. The results indicated by the reagents can be determined by comparing the reagent patches 769 with the coloured patches 764.

The coloured patches 764 are located near the reagent patches 769 and are for interpreting or reading the results indicated by the reaction of the reagents to the specimen. For example, the pH reagent strip may indicate colours ranging from orange through yellow and green to blue. Accordingly, the coloured patches 764 located in this vicinity are for coloured to enable a comparison that would enable a determination of the pH of the specimen. Similarly, the colours indicated by the protein reagent patch range from yellow for “Negative” through yellow-green and green to green-blue for “Positive” and the adjacent coloured patches will be coloured to enable a comparison that would enable a determination of a negative or a positive reaction. The colours indicated by the nitrite reagent patch are different intensities of pink and the adjacent coloured patches will be coloured to enable a comparison that would enable a determination of the nitrite concentration. Likewise, the colours indicated by the leukocyte reagent patch are different intensities of purple and the adjacent coloured patches will be coloured to enable a comparison that would enable a determination of the esterase activity.

In an embodiment, the reagent patches 769 may be comprised of a reagent that provides a binary response. That either a positive or a negative response. For example, the pH reagent strip may indicate one colour indicating pH in the range of 5.0-7.5 and another colour indicating pH in the range of 7.5-9. The protein test strip may indicate one colour to indicate a range of negative or trace and another colour indicating a positive response. The nitrite test strip may indicate one colour to indicate a range of negative or trace and another colour indicating a positive response. The Leukocyte esterase test strip may indicate one colour to indicate a range of negative or trace and another colour indicating a positive response. In such an embodiment, because the reagent test strips indicate a binary response, the cap 765 may still include a circular array of coloured patches 764 or these may be done away with altogether.

Bacterial infection is indicated where pH is indicated in the range of 7.5-9, protein is indicated in the range of ++ or +++, nitrite is indicated in the range ++ or +++ and Leukocyte esterase is indicated in the range ++ or +++.

The apparatus 701 of FIGS. 20 to 24 is advantageous in that it provides an easy to use, hygienic, and relatively accurate device for collecting a nasal secretion specimen, contacting reagents with the specimen and interpreting results indicated by the colour or change in colour of the reagents for diagnosing a bacterial sinus infection.

Advantageously, with the same combination of reagents it is possible to diagnose other suspected causes of sinusitis including allergic rhinitis or upper respiratory tract viral infection. Allergic rhinitis is indicated where pH is indicated in the range of 5.0-7.5, protein is indicated in the range of Trace to +, nitrite is indicated in the range Negative or Trace and Leukocyte esterase is indicated in the range Negative or Trace. Viral infection is indicated where pH is indicated in the range of 7.5-9, protein is indicated in the range of Trace to +, nitrite is indicated in the range Negative or Trace and Leukocyte esterase is indicated in the range Trace to +.

In an embodiment, an image capturing device such as a mobile device or smartphone may be employed to take an image of the circular array of coloured patches 764 and the reagent patches or strips 769 through the transparent base plate 763. The image data can be processed to determine automatically the results indicated by the reagents. In an embodiment, computer vision is used to interpret the colour reagent responses into quantitative and qualitative clinical data. In another embodiment, dedicated imaging hardware may be employed that includes a pre-calibrated scanner, which is operated in well-known and monitored illumination conditions, and a classifier that operates based on the calibrated images derived by the scanner.

In embodiments, the diagnostic element may be comprised of a biosensor for the detection of one or more of the target chemical substances. The biosensor may include a biological component and a physicochemical detector. The biological component may include one or more enzymes or antibodies to target an analyte such as any one or more of those described herein. The transducer or detector is adapted for detecting a reaction between the target analyte and the biological component. The transducer may be comprised of an optical, piezoelectric, electrochemical or an electro-chemiluminescence detector.

In other embodiments, the diagnostic element may comprise a DNA-based assay such as a polymerase chain reaction (PCR) assay to identify a target pathogen. The PCR assay can be a device for performing real-time polymerase chain reaction (real-time PCR) such as a miniaturized PCR module.

Referring to FIGS. 25 and 26, there is shown another apparatus 801 for the collection of a nasal secretion sample and for the diagnosis of any one or more of a bacterial sinus infection, a viral infection, a fungal infection, an allergic immune response and an inflammatory response from the sample in accordance with another embodiment.

Apparatus 801 includes a device 810 that in many respects is similar to the device 710 of FIGS. 20 to 24 except that the peripheral wall 832 terminates at an upper edge 838 that has a flat profile. The apparatus 801 includes a different form of the cap 865 that is configured for use with a lateral flow assay device 880 that is configured to indicate the presence or absence of any one or more of a bacterial sinus infection, a viral infection, a fungal infection, an allergic immune response and an inflammatory response from a nasal secretion specimen.

The cap 865 includes a base 863 and a cylindrical body 867 upstanding therefrom. The cap 865 is preferably formed out of a clear plastic material. The base 863 includes a central channel or port 864 through the base 863. The cylindrical body 867 has an internal cylindrical shaped recess 868 defined by a cylindrical shaped surface 870. As illustrated in FIG. 25, the cap 865 is fitted to the collection device 810 by inserting the cylindrical body 867 into the aperture 826 of the housing 820 in an interference or snap fit. The cylindrical recess 868 provides room for the protuberance 840 therewithin.

When the cap 865 fitted to the collection device 810 the port 864 is inserted into an opening 884 in the lateral flow assay device 880. The collection device 810, cap 865 and the lateral flow assay device 880 are thereby assembled and the entire assembly is supported upright on the lateral flow assay device 880. Under the action of gravity, the nasal secretions in the reservoir pass back through the apertures in the base and into the cylindrical recess 868 of the cap 865. The nasal secretions continue to flow through the port 864 and into the opening 884 of the lateral assay device 880. One or more reagents within the lateral flow assay device 880 react with the nasal secretions and provide a visual indication in an indication window 885 of the presence or absence of any one or more of a bacterial sinus infection, a viral infection, a fungal infection, an allergic immune response and an inflammatory response from the nasal secretion specimen.

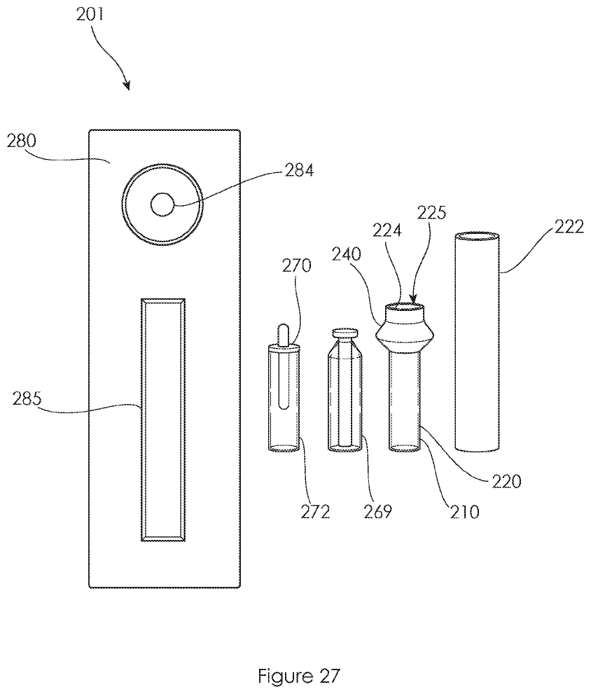

FIG. 27 illustrates another apparatus 201 for the collection of a nasal secretion sample and for the diagnosis of any one or more of a bacterial sinus infection, a viral infection, a fungal infection, an allergic immune response and an inflammatory response from the sample in accordance with another embodiment.

The apparatus 201 includes a device 210 for the collection of a nasal secretion specimen in accordance with an embodiment of the invention. The device 210 includes a cylindrical housing 220 including a reservoir 225 and an opening 224 at one end. A silicon rubber fitting 240 is attached to the open end of the housing 220. The fitting 240 is adapted for insertion into the nostril and to provide a substantial seal between the nostril and the reservoir 225. A handle member 222 comprised of a flexible tube is adapted for connection with the housing 220 to provide a gripping surface for a user to hold while inserting the housing 220 into the nostril.

The apparatus 201 further includes a reagent container 269 and pipette 270 adapted to transfer reagent from the container 269 to the reservoir 225 containing the specimen. The silicon rubber fitting 240 is removed from the housing 220 before transferring the reagent. The pipette 270 is configured to seal the reservoir 225 opening 224 as illustrated in the exemplary additional sealed reservoir 272. After a period of time elapses, the pipette 270 is used to transfer some of the specimen reagent mixture into an opening 284 in a lateral flow assay device 280. After a period of time, an indication is provided in an indication window 285 of the assay device 280 of the presence or absence of any one or more of a bacterial sinus infection, a viral infection, a fungal infection, an allergic immune response and an inflammatory response from the nasal secretion specimen.

FIGS. 28 to 30 illustrate another apparatus for the collection of a nasal secretion sample and for the diagnosis of any one or more of a bacterial sinus infection, a viral infection, a fungal infection, an allergic immune response and an inflammatory response from the sample in accordance with another embodiment.

The apparatus includes a specimen collection device 310 in the form of a card 320 which has thumb hold zones 322, 324 on left and right sides and sample zones 323, 325 that are marked 1′ and ‘R’ that are contacted with respective nostrils. The card includes tear lines 327, 328, 329 between the zones 322, 323, 324, 325.

The patient uses the thumb hold zones 322, 324 to grip and hold the sample zones 323, 325 against the nostrils while depositing nasal secretions onto the sample zones 323, 325. The sample zones 323, 325 may comprise an absorbent material such as a sponge material to absorb a specimen of the nasal secretion. The sample zones 323, 325 are separated by tearing apart along the tear lines 327, 328, 329.

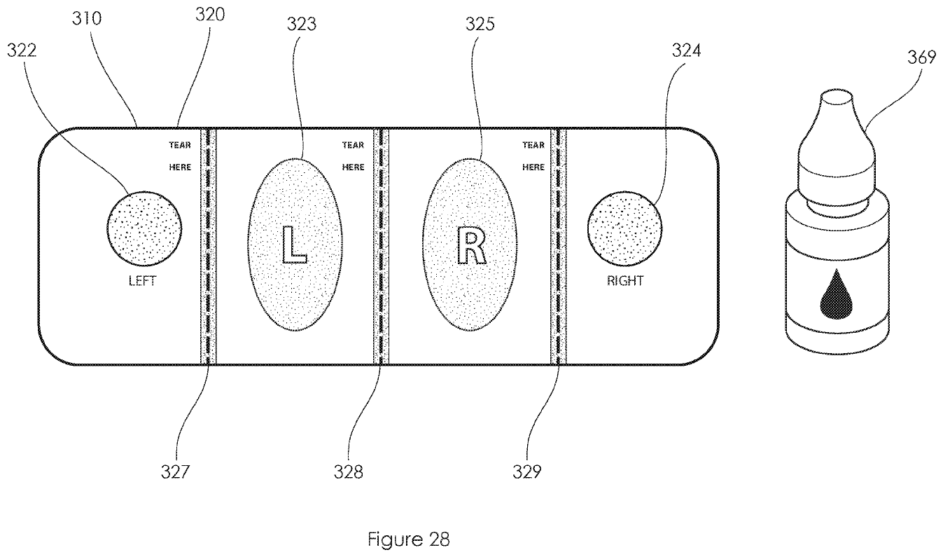

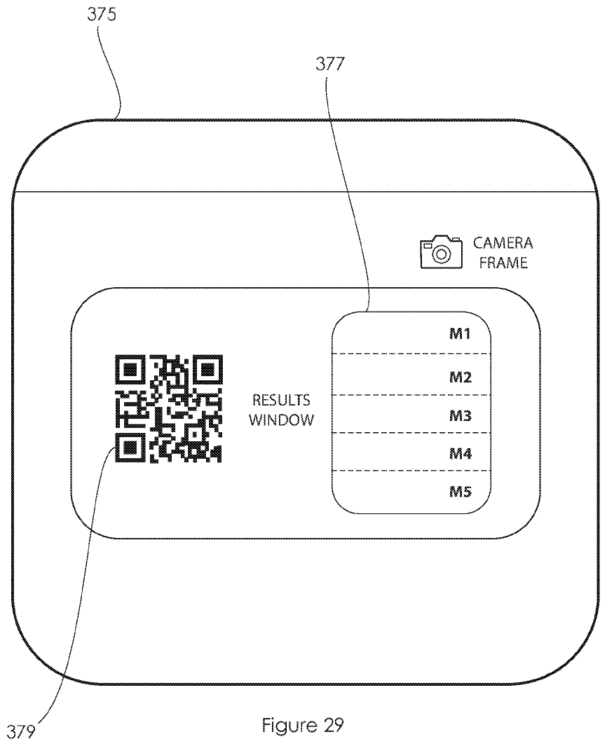

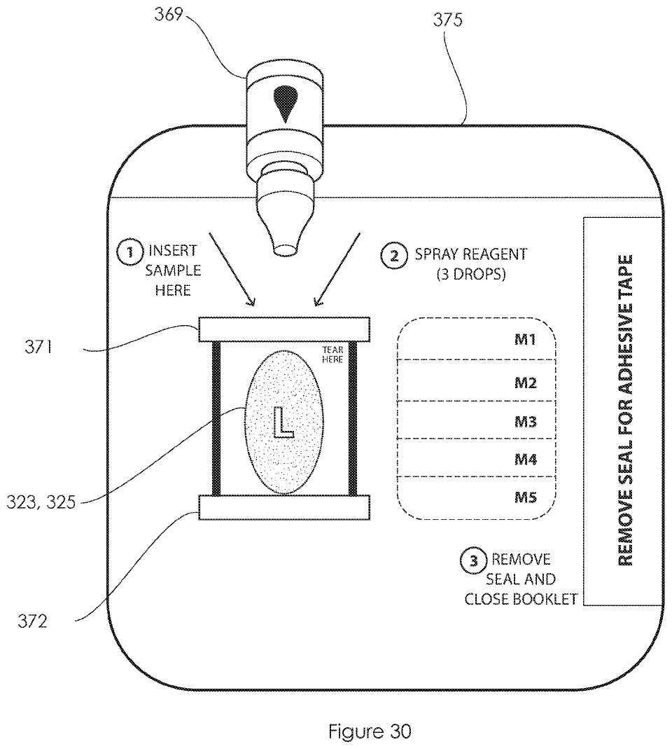

As illustrated in FIGS. 29 and 30, the apparatus 301 further includes a test card 375. FIG. 30 illustrates the test card in an open condition. One of the separated sample zones 323, 325 is applied to the test card 375 and is retained in place by retention members 371, 372 and reagent is applied to the sample zone 323, 325 from a reagent container 369. The test card is then closed as indicated in FIG. 29. After a period of time has elapsed, the specimen and reagent mixture will provide an indication in the results window 377. Using a smartphone or tablet, a machine-readable code 3793, such as a QR code, may be scanned and a digital image taken of the results window 377 and transmitted for processing and analysis. A result is then transmitted back to the smartphone or tablet or otherwise with a diagnosis of the presence or absence of any one or more of a bacterial sinus infection, a viral infection, a fungal infection, an allergic immune response and an inflammatory response infection from the nasal secretion specimen. In embodiments, apparatus 301 can also diagnose allergic rhinitis or upper respiratory tract viral infection.

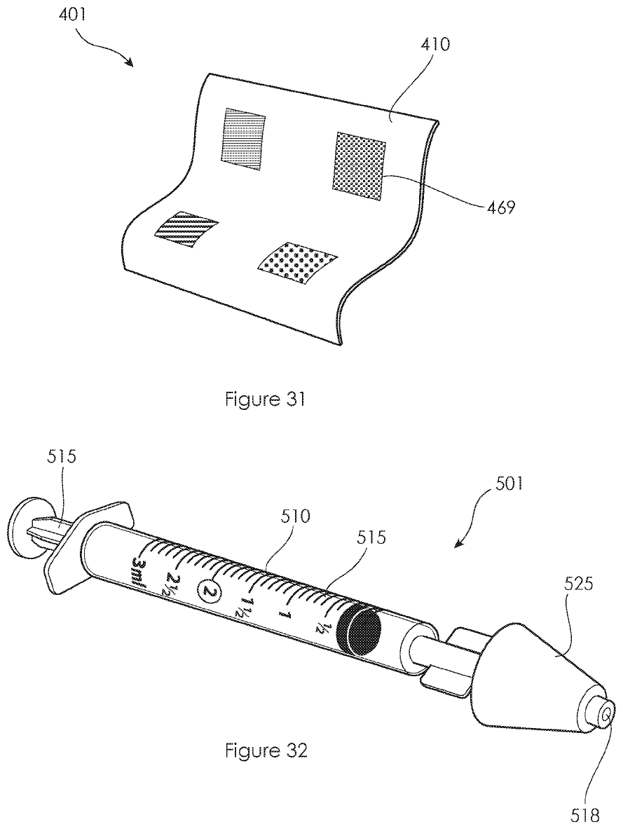

FIG. 31 illustrates another apparatus 401 for the collection of a nasal secretion sample and for the diagnosis of any one or more of a bacterial sinus infection, a viral infection, a fungal infection, an allergic immune response and an inflammatory response from the sample in accordance with another embodiment. The apparatus 401 include a specimen collection device 410 comprised of a sheet of flexible absorbent material. Reagent patches 469 are provided at different locations on the device 410. In use, the device is contacted with the nostrils and a specimen of nasal secretion is deposited onto the device 410. The specimen is absorbed into the material and contacts with the reagent patches. When a period of time has elapsed for the reagents to react with the nasal secretions the reagent patches can be compared with an array of coloured patches (not shown) or otherwise analysed to determine if an indication is provided of the presence or absence of any one or more of a bacterial sinus infection, a viral infection, a fungal infection, an allergic immune response and an inflammatory response from the nasal secretion specimen.

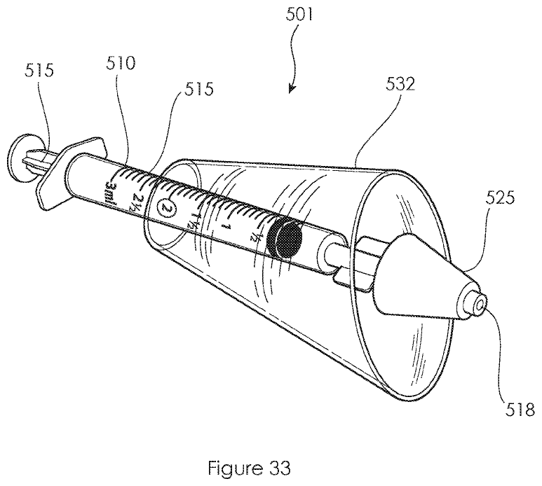

FIGS. 32 and 33 illustrate another apparatus 501 for the collection of a nasal secretion sample and for the diagnosis of any one or more of a bacterial sinus infection, a viral infection, a fungal infection, an allergic immune response and an inflammatory response from the sample in accordance with another embodiment.

The apparatus 501 includes a device 510 for the collection of a nasal secretion. The device 510 consists of a syringe 515 including a cylinder 516 containing a solution, a plunger 515 and an outlet 518. A cone shaped foam tip 525 surrounds the outlet 518 of the syringe for insertion into the nostril. Depressing the plunger 515 causes the solution to be ejected from the outlet 518 and into the nostril to irrigate the nasal cavity. The foam tip 525 absorbs a sample consisting of a nasal secretion specimen and the irrigation solution.

FIG. 33 illustrates an embodiment of the device 510 further including a shroud 532 coupled to the cylinder and adapted to surround, at least in part, the outlet 518 and the foam tip 525. The shroud 532 operates to contain nasal secretions and irrigation solution, or at least prevent them from being spread uncontrollably.

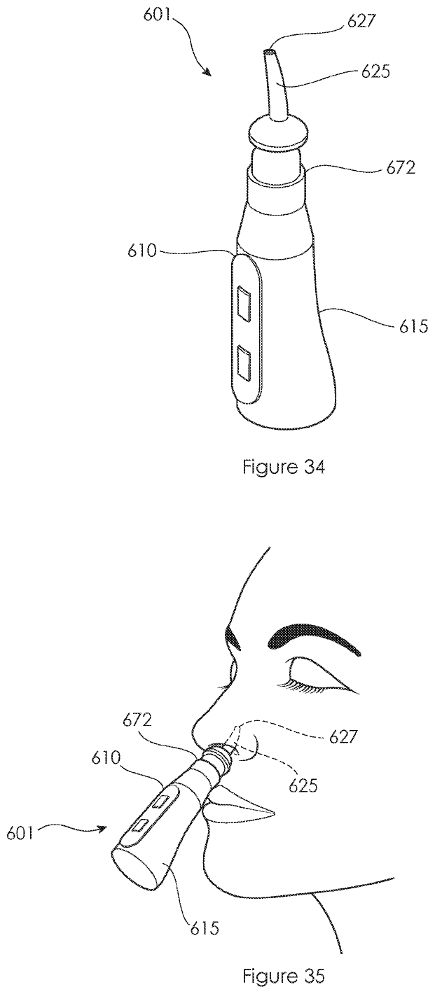

FIGS. 34 and 35 illustrate another apparatus 601 for the collection of a nasal secretion sample and for the diagnosis of any one or more of a bacterial sinus infection, a viral infection, a fungal infection, an allergic immune response and an inflammatory response from the sample in accordance with another embodiment.

The apparatus 601 includes a device 610 for the collection of a nasal secretion. The device 610 consists of a main housing 615 containing a mechanical vacuum or suction device, which may be battery powered of manually powered. The apparatus also includes a tip member 625 including a base housing 672 and a hollow tube 627 extending from the base housing 672. The base housing 672 contains a reservoir therewithin which is in fluid communication with the hollow tube 627. The base housing 672 is adapted to be mounted to the main housing 615 so that the reservoir and/or the hollow tube 627 are in fluid communication with the suction device.

The tip member 625 is adapted for insertion into a nostril and operation of the vacuum device causes suction to be applied through the hollow tube 627 of the tip member 625 to draw nasal secretions into the hollow tube 627. The nasal secretions are conveyed into the reservoir within the base housing 672 at the base of the tip member 625. A one-way valve permits flow of nasal secretions into the reservoir and prevents the reverse flow of the secretions out of the reservoir and into the hollow tube 627.

The apparatus 601 also includes an assay, such as any one of the assays described herein. In one embodiment, the base housing 672 includes a store of a reagent that is exposed to the sample contained in the reservoir and an assay device in which after a period of time, an indication is provided in an indication window of the assay device of the presence or absence of any one or more of a bacterial sinus infection, a viral infection, a fungal infection, an allergic immune response and an inflammatory response from the nasal secretion specimen. In another embodiment, the base housing 672 includes one or more reagent patches that are exposed to the sample contained in the reservoir. The reagent base housing 672 may include a window so that the patches are visible. The base housing 672 may also include an array of coloured patches for reading the results of the reagent patches.

The tip member 625 may be disposable and the main housing 615 containing the mechanical vacuum or suction device may be reusable. The embodiment of FIGS. 34 and 35 is advantageous as it may be used with babies, young children or disabled patients who may not have the cognitive ability to expel a nasal secretion sample on demand.

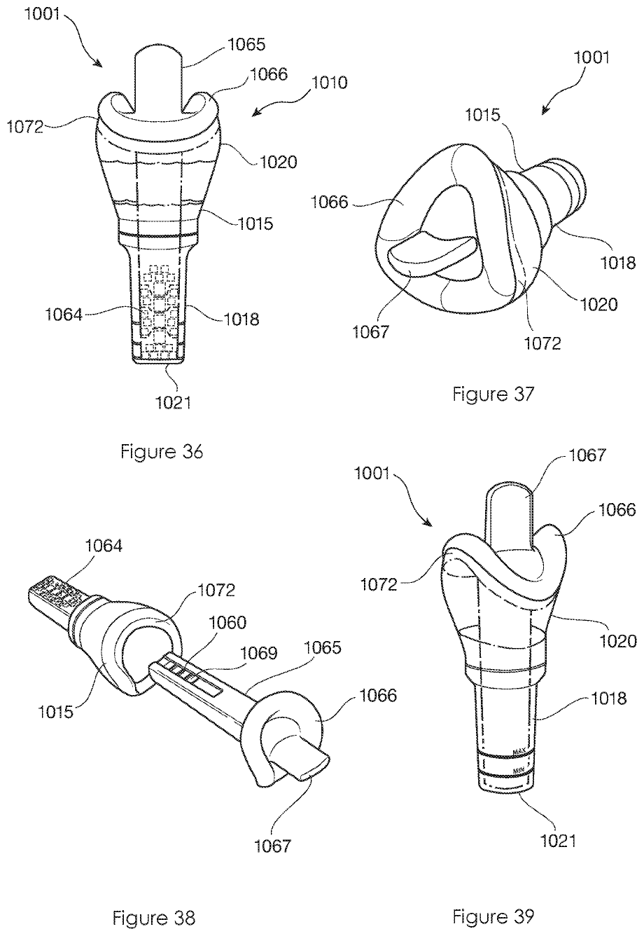

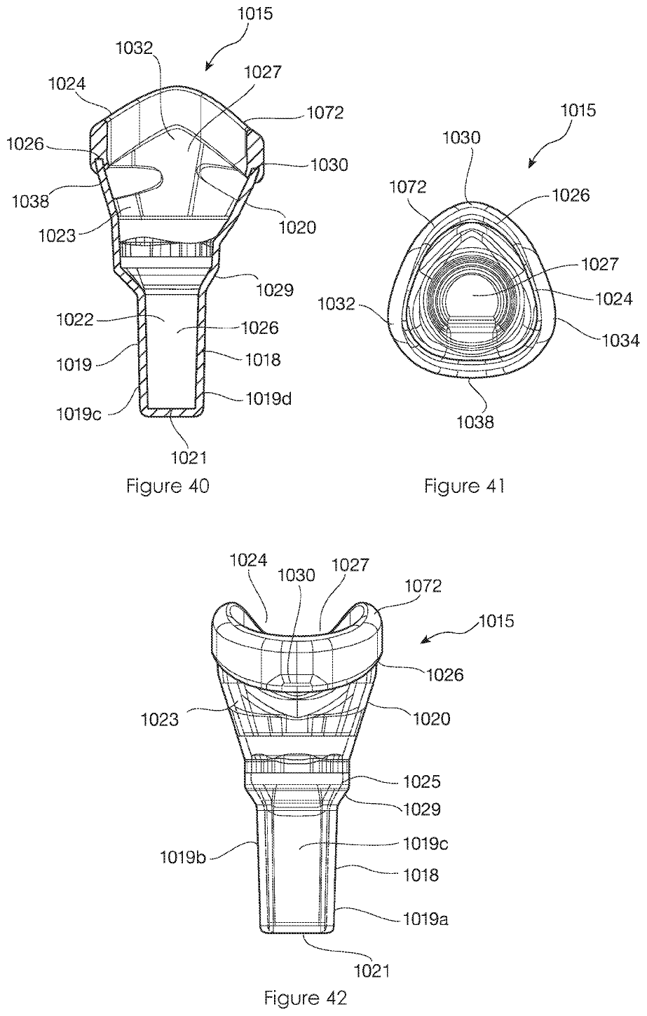

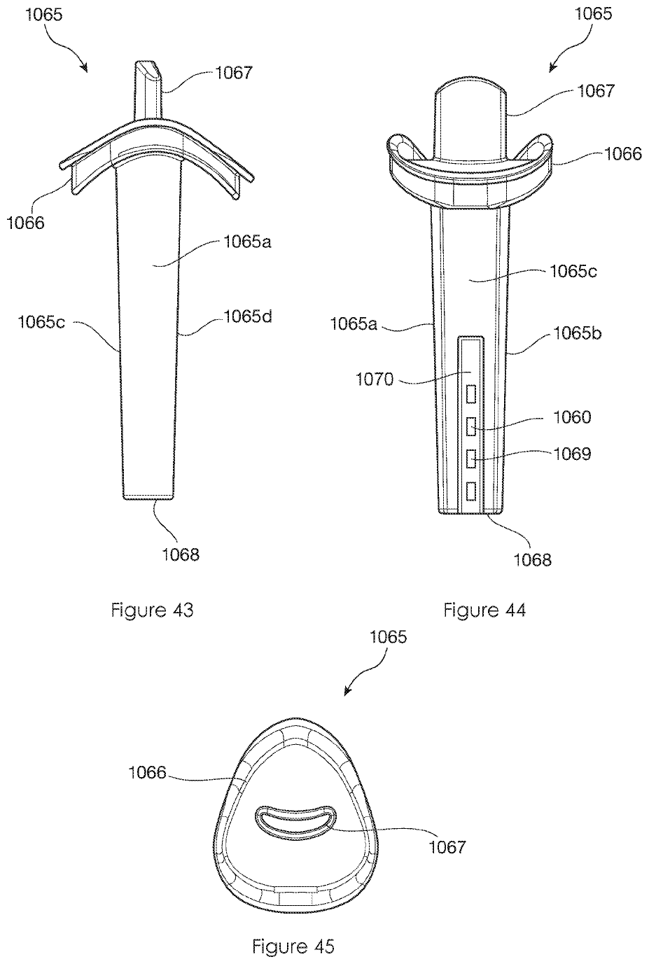

FIGS. 36 to 45 illustrate an apparatus 1001 for the collection of a nasal secretion sample and for the diagnosis of any one or more of a bacterial sinus infection, a viral infection, a fungal infection, an allergic immune response and an inflammatory response from the sample. The apparatus includes a device 1010 for the collection of a nasal secretion specimen in accordance with an embodiment of the invention. The device 1010 includes a housing 1015 having an elongated reservoir section 1018 and a funnel shaped section 1020.

The reservoir section 1018 has a closed base 1021 and a wall 1019 upstanding therefrom. The closed base 1021 and the wall 1019 together define an interior cavity 1022 that functions as a reservoir 1026 for receiving a nasal mucus sample. It is to be appreciated that the reservoir section 1018 and the wall 1019 can have a circular cross section, a rectangular cross section, an oval shaped cross section or one of a number of other polygonal cross sections. In the illustrated embodiment, the wall 1019 has a cross section that is substantially rectangular albeit with a pair of opposite curved short sides 1019a, 1019b and a pair of opposite flat long sides 1019c, 1019d. The curved sides 1019a, 1019b and the flat sides 1019c, 1019d are elongated and extend from the base 1021 to an opposite open end 1029 at which the reservoir section 1018 adjoins the funnel shaped section 1020. The open end 1029 is wider than the base 1021 and the curved sides 1019a, 1019b and the flat sides 1019c, 1019d progressively taper from the wider open end 1029 to the narrower base 1021.

The funnel shaped section 1020 is comprised of a shell shaped wall 1023 that is dimensioned to receive a nose of a person therewithin. The shell shaped wall 1023 has a central opening 1025 adjoining the open end 1029 of the reservoir section 1018. The wall 1023 extends about and from the central opening 1025 to a free edge 1026 that defines an open end 1024 of the funnel shaped section 1020. The shell shaped wall 1023 defines an internal cavity 1027 for receiving the person's nose through the open end 1024 and to direct a nasal secretion sample into the reservoir 1026.

The wall 1023 of the funnel shaped section 1020 comprises an apical section 1030, lateral sections 1032, 1034 and a bottom section 1038. The apical section 1030 fits over the nose and seals against the bridge of the nose. The lateral sections 1032, 1034 are adapted to seal against the upper cheek area of the person's face on both sides of the nose. The bottom section 1038 is adapted to seal against and across the top lip beneath the wearer's nose. Accordingly, when viewed from a side elevation with the open end 1024 of the funnel shaped section 1020 in an upward facing orientation, the lateral sections 1032, 1034 each have a generally triangular shaped form with a base and side edges tapering to an apex. The base of each lateral section 1032, 1034 extends between the apical section 1030 on one side and the top lip engaging section 1038 on the other side.

A flexible and resilient cushion 1072 is attached to the free edge 1021 of the funnel shaped section 1020 and is adapted to contact and seal against the face of the wearer. The flexible and resilient cushion 1072 may be formed of a resilient polymer such as a soft silicone elastomer or a resilient foam material. The housing 1015, including the reservoir section 1018 and the funnel shaped section 1020, is preferably formed of an injection moulded clear or at least transparent polycarbonate material. The cushion 10722 may be formed to include a slot adapted to receive the free edge 2021 or may be overmoulded onto the funnel shaped section 1020.

The apparatus 1001 further includes an elongated stick 1065 adapted for insertion into the reservoir section 1018. At one end the elongated stick 1065 includes a gripping portion 1067 for a user to hold such as between the thumb and forefinger. At the other end the elongated stick 1065 supports a diagnostic element 1060 and is adapted for insertion into the reservoir 1026. The end of the stick 1065 supporting the diagnostic element 1060 is shaped complementarily with the shape of the reservoir 1026. In the illustrated embodiment, the elongated stick 1065 has a cross section that is substantially rectangular albeit with a pair of opposite curved short sides 1065a, 1065b and a pair of opposite flat long sides 1065c, 1065d. The curved sides 1065a, 1065b and the flat sides 1065c, 1065d are elongated and taper from the gripping portion 1067 to a substantially planar tip 1068 in a similar fashion to the reservoir 1026.

A disc shaped shield 1066 is provided between the gripping portion 1067 and the end of the stick 1065 supporting the diagnostic element 1060.

One of the flat sides 1065c, 1065d supports the diagnostic element 1060 which in the illustrated embodiment is comprised of reagent patches or strips 1069 provided thereon. The side supporting the diagnostic element has an elongated channel or recess 1070 configured to receive the patches or strips 1069. The one or more reagents are selected from the group including indicators of pH, protein content, nitrite content, leukocyte esterase activity, and eosinophil content. Such reagent strips may include those that are commonly employed for testing urine, or ‘urine dipsticks’, to diagnose UTI, diabetes and kidney disorders. Exemplary reagents include: pH: methyl red and bromothymol blue; Protein: tetrabromophenol blue; Nitrite: p-arsanilic acid; 1,2,3,4-tetrahydrobenzo-(h)-quinolin-3ol; Leukocytes: derivatized pyrrole amino acid ester; diazonium salt.

When a nasal secretion sample is deposited into the reservoir 1026 and the elongated stick 1065 is subsequently inserted into the reservoir 1026 the elongated stick 1065 displaces the sample. The curved sides 1065a, 1065b and the flat sides 1065c, 1065d and the tip 1068 of the stick 1065 are dimensioned slightly smaller than the corresponding curved sides 1019a, 1019b and flat sides 1019c, 1019d and base 1021 of the reservoir section 1018 such that a relatively small gap (1-3 millimetres) remains therebetween. The nasal secretion sample is thereby displaced by the elongated stick 1065 into the space between the exterior surface of the elongated stick 1065 and the interior surface of the reservoir 1026.

In an embodiment, the nasal secretion sample is displaced into the elongated channel or recess 1070 containing the patches or strips 1069. The stick 1065 may be dimensioned for a relatively close fit within the reservoir section 1018 such that the only space into which the nasal secretion sample can be displaced within the reservoir section 1018 is the elongated channel or recess 1070. Thus, the entire diagnostic element 1060, which in the illustrated embodiment comprises reagent patches or strips 1069, is contacted with the nasal secretion sample.

The disc shaped shield 1066 functions to seal the reservoir 1026 or to seal against the cushion 1022 on the free edge 1021 of the funnel shaped section 1020. The collection device 1010 including the entire assembly of the housing 1015 and the elongated stick 1065 is stood upright on the base 1021 and left for a period for the nasal secretions to react with the reagent patches or strips 69.

One of the planar or flat long sides 1019c, 1019d of the wall 1019 is transparent to allow light to reach the reagent patches or strips 1069 on the elongated stick 1065. The same one of the flat long sides 1019c, 1019d is provided with a series of coloured patches 1064 that are positioned to be immediately adjacent to a corresponding reagent patch 1069 when the elongated stick 1065 is fully inserted into the reservoir 1026. In this way, the coloured patches 1064 and the reagent patches 1069 can both be viewed at the same time and side by side without needing to withdraw the elongated stick 1065. The results indicated by the reagents can be determined by comparing the reagent patches 1069 with the coloured patches 1064.

Accordingly, the coloured patches 1064 are for interpreting or reading the results indicated by the reaction of the reagents 1069 to the specimen. For example, the pH reagent strip may indicate colours ranging from orange through yellow and green to blue. Accordingly, the coloured patches 1064 located in this vicinity are coloured to enable a comparison that would enable a determination of the pH of the specimen. Similarly, the colours indicated by the protein reagent patch range from yellow for “Negative” through yellow-green and green to green-blue for “Positive” and the adjacent coloured patches will be coloured to enable a comparison that would enable a determination of a negative or a positive reaction. The colours indicated by the nitrite reagent patch are different intensities of pink and the adjacent coloured patches will be coloured to enable a comparison that would enable a determination of the nitrite concentration. Likewise, the colours indicated by the leukocyte reagent patch are different intensities of purple and the adjacent coloured patches will be coloured to enable a comparison that would enable a determination of the esterase activity. Other reagent types described herein with reference to other embodiments may also be employed in the embodiment of FIGS. 36 to 45.

In another embodiment, the diagnostic element 1060 supported by the stick 1065 is comprised of an embedded diagnostic lateral flow immunochromatographic assay (i.e. for viral detection such as influenza). The lateral flow assay (LFA) is a paper-based platform for the detection and quantification of analytes in complex mixtures, where the sample is placed on a test device and the results are displayed within 5-30 mi

In another embodiment, the diagnostic element 1060 is an embedded electrochemical detection microassay chip. In another embodiment, the diagnostic element 1060 is a biosensor or other bioassay. In another embodiment, the diagnostic element 1060 is a miniaturised DNA amplification (PCR lab on chip) device.

In embodiments, the diagnostic element 1060 is located internally within the stick and insertion of the elongated stick 1065 into the reservoir 1026 displaces the sample and forces the sample into a fluid passageway within the elongated stick 1065 to deliver the sample to the internally located diagnostic element.

The apparatus 1001 of FIGS. 36 to 45 is advantageous in that it provides an easy to use, hygienic, and relatively accurate device for collecting a nasal secretion specimen, contacting reagents with the specimen and interpreting results indicated by the colour or change in colour of the reagents for diagnosing a pathology such as any one or more of a bacterial sinus infection, a viral infection, a fungal infection, an allergic immune response and an inflammatory response. In embodiments, it is possible to diagnose other suspected causes of sinusitis including allergic rhinitis or upper respiratory tract viral infection.

Although the invention has been described with reference to specific examples, it will be appreciated by those skilled in the art that the invention may be embodied in many other forms, in keeping with the broad principles and the spirit of the disclosure described herein.

1-32. (canceled)

33. A respiratory secretion sample collection device, including:
a collection reservoir for directly receiving a sample of a respiratory secretion;
a displacement member for insertion into the collection reservoir and displacing the sample within the collection reservoir;
a container of a diluent for fluid communication with the sample for mixing the diluent with the sample,
an outlet for discharging the mixture of the diluent and the sample to an assay device.

34. The collection device of claim 1, wherein the displacement member comprises an elongated member including a resilient seal for sealing against an internal wall of the collection reservoir.

35. The collection device of claim 2, wherein the resilient seal deforms upon insertion into the collection reservoir to provide a tight seal against the internal wall of the collection reservoir substantially throughout a complete stroke for displacing substantially all of the sample in the collection reservoir.

36. The collection device of claim 1, wherein the collection device includes a cavity having a predetermined volume wherein insertion of the displacement member into the collection reservoir displaces the sample into the cavity for measuring out a predetermined volume of the sample.

37. The collection device of claim 4, wherein the cavity is disposed within the displacement member and includes an inlet opening for fluid communication with the collection reservoir for the displaced sample to enter the cavity.

38. The collection device of claim 5, wherein the displacement member includes an internal space and the channel includes an outlet opening in fluid communication with the internal space for the sample to overflow into the internal space.

39. The collection device of claim 6, wherein the outlet opening of the cavity is adapted for fluid communication with the container of the diluent.

40. The collection device of claim 7, wherein the container of the diluent includes an opening having a seal that is pierced by a protrusion adjacent to the outlet opening of the cavity by coupling the diluent container with the cavity.

41. The collection device of claim 1, wherein the collection reservoir includes a void internal volume comprising a sample inlet opening and a resilient shroud adjacent to the sample inlet opening for providing a partial seal around a patient's nose.

42. The collection device of claim 4, wherein the collection reservoir includes an outlet opening for fluid communication with the cavity upon insertion of the displacement member into the reservoir.

43. The collection device of claim 10, wherein the outlet opening of the collection reservoir includes a seal for directing the sample into the cavity by insertion of the displacement member into the collection reservoir.

44. The collection device of claim 11, wherein the seal for the outlet opening of the collection reservoir is pierced by connection of the collection reservoir with a mixing chamber.

45. The collection device of claim 12, wherein the mixing chamber includes an inlet opening and a protrusion adjacent thereto for piercing the seal of the outlet opening of the collection reservoir and for fluid communication between the container of diluent, the cavity and the mixing chamber.

46. The collection device of claim 13, wherein the diluent container includes a reciprocating plunger for displacing the sample in the cavity and the diluent between the diluent container and the mixing chamber for mixing the sample and the diluent.

47. The collection device of claim 12, wherein the outlet for discharging the mixture of the diluent and the sample to an assay device includes an outlet of the mixing chamber to the assay device.

48. The collection device of claim 1, wherein the diluent container includes graduated markings for controlling a volume of the diluent to be mixed with the sample and for controlling a volume of the mixture of the diluent and the sample discharged to the assay device.

49. The collection device of claim 4, wherein the collection reservoir includes a closed end for directing the sample into the cavity upon insertion of the displacement member into the reservoir.

50. The collection device of claim 4, wherein the diluent container includes an internal chamber in fluid communication with the cavity for mixing the sample and the diluent.

51. The collection device of claim 12, wherein the assay device includes a lateral flow assay element within a housing adapted for insertion into the mixing chamber and for displacing the mixture of the diluent and the sample into contact with the assay element within the housing.

52. A method for the collection of a respiratory secretion sample and for the detection of biomarkers indicative of any one or more of a viral respiratory infection, a bacterial sinus infection, a fungal infection, an allergic immune response and an inflammatory response, including:
collecting a respiratory secretion sample directly into a collection reservoir;
displacing the sample within;
mixing the sample with a diluent;
discharging the mixture of the sample and the diluent to an assay device for determining the presence of biomarkers in the mixture indicative of any one or more of a viral respiratory infection, a bacterial sinus infection, a fungal infection, an allergic immune response and an inflammatory response.
